(19) United States
(12) Patent Application Publication
(10) Pub. No.: US 2004/0166559 A1
(43) Pub. Date: Aug. 26, 2004

(54) Vectors for gene therapy

(76) Inventors: Jane F. Apperley, London (GB); Marina I. Garin, London (GB)

Correspondence Address: NIKOLAI & MERSEREAU, P.A., 900 SECOND AVENUE SOUTH, SUITE 820, MINNEAPOLIS, MN, 55402, US

(21) Appl. No.: 10/257,309

(22) Filed: Oct. 10, 2002

(30) Foreign Application Priority Data
Apr. 13, 2001 (WO) PCT/GB01/01640

(51) Int. Cl. C07H 21/04; C12N 9/12

(52) U.S. Cl. 435/69.1; 435/194; 435/320.1; 435/325; 536/23.2

(57) ABSTRACT

A polynucleotide encoding a thymidine kinase wherein the thymidine kinase coding region does not contain a functional splice acceptor and/or splice donor site. An expression vector comprising said polynucleotide. The polynucleotides and expression vectors are useful in gene therapy. The polynucleotides and vectors are useful in destroying cells when used in conjunction with a substantially non-toxic agent, such as ganciclovir, which is converted by thymidine kinase to a toxic agent.

The present invention relates to vectors for gene therapy. In particular it relates to vectors for gene therapy which encode the thymidine kinase gene, and more particularly it relates to retroviral vectors encoding this gene.

In cancer gene therapy there is considerable interest in the use of metabolic suicide genes for the treatment of tumours. Transfer of suicide genes into tumour cells that are not normally expressed in mammalian cells can activate non-toxic prodrugs to their cytotoxic form. This would induce tumour regression by killing of the engineered tumour cells (Mullen 1994, Pharmacol. Therapeut. 63, 199-207). The HSV-tk/GCV system is one of the most widely used approaches in suicide gene therapy (Moolten F L, Wells J M, Cancer Res. 46: 5276, 1986; Moolten F L, Cancer Gene Ther. 1: 279, 1994). Cells expressing the HSV-tk gene can phosphorylate GCV and eventually leading to cell death as a result of interfering with the ability to replicate DNA. This approach is currently used in more than 30 clinical trials for gene therapy of a variety of human cancers (Ross et al, 1996; Hum. Gene Ther.:7, 1782-90).

Allogeneic bone marrow transplantation (allo-SCT) is widely used as a curative approach to many haematological malignancies. The success of allo-SCT however is limited by a number of factors, including the clinical entity of graft versus host disease (GvHD) mediated by immunocompetent donor derived T-lymphocytes. Strategies for the prevention of GvHD include the use of immunosupression following transplantation and/or ex vivo or in viva T-cell depletion of the graft. The first is only partially successful in the prevention of GvHD and may contribute to a delay in immune reconstitution, thus resulting in considerable morbidity and mortality. The latter is associated with an increase in the incidence of both graft rejection and leukaemic relapse. In some cases recurrence may be successfully treated by further infusions of donor lymphocytes (DLI; Kolb H J et al: Blood 86: 2041, 1995), but these may also result in severe and potentially fatal GvHD.

Recently the use of the HSV-tk/GCV system has been proposed for the specific and conditional ablation of alloreactive donor T-cells after allogeneic hematopoietic stem cell transplantation (Bordignon C et al Hum. Gene Ther. 6: 813, 1995; Tiberghien P, et al Hum. Gene Ther. 8: 615, 1997; Link et al, Human Gene Ther. 1998, 9, 115-134). This has the potential to modulate the graft versus host disease (GvHD) while preserving the graft versus leukaemia effect (GvL).

Donor T-cells expressing HSV-tk have been used in the prevention and management of both leukaemic relapse and Epstein-Barr virus associated lymphoproliferative disorders after allo-SCT. In a clinical study, GvHD induced by these donor cells responded to GCV (Bonini C, et al Science 276: 1719, 1997). However in this study one patient showed partial resistance to GCV-mediated depletion of transduced donor T-cells. GCV resistance in HSV-tk transduced cells is a matter of concern because this may limit the efficacy of this approach. This clinical observation confirms data available from pre-clinical studies. Using the wild-type HSV-tk gene, the rate of inhibition of cell proliferation after GCV treatment ranges between 80% and 90%. Complete eradication of the genetically engineered tumour cells can not be achieved in many instances. Cells resistant to GCV treatment have been found within the HSV-tk transduced populations. In some cases, a higher dose of GCV has been used to induce cell death. However, this approach may not be applicable in the clinical situation as the GCV toxicity in humans is cumulative. Despite the great expectations generated with these novel approaches, optimised versions of HSV-tk gene could help to circumvent some of these limitations. Ideally, enhanced efficiency (100% killing) and improved efficacy (lower doses of GCV required) are desirable for the successful use of the HSV-tk/GCV system. Such differences are likely to affect the choice of the suicide gene for clinical use.

During our studies on genetically modified donor T lymphocytes, we have now identified at least one cause of GCV resistance in HSV-tk transduced cells. Unexpectedly we have found that in the retrovirus producer cells, part of the tk mRNA derived from the provirus becomes spliced in the vector-derived mRNA. This is believed to be due to the presence of nucleotide sequences in the tk mRNA which act as splice sites (which may be termed “cryptic splice sites”) to cause the production of a small proportion of virus particles carrying the aberrant form of the HSV-tk gene, the remainder containing the full length form. This mechanism explains the passage of the truncated provirus HSV-tk gene to the target cells. We have prepared mutants of the thymidine kinase gene in which the splice sites are removed, and which do not lead to the production of the aberrant form of the thymidine kinase gene. These lead to a greater proportion of the transduced target cells correctly expressing thymidine kinase.

Mutants in the thymidine kinase gene have been made which increase the biological (enzymatic) activity. For example, Kokoris et al (1999) Gene Therapy 6, 1415-1426 described the production and screening of a large library of mutant HSV-tk genes for enzymes with an ability to enhance in vitro cell sensitivity to GCV and acyclovir (ACV). The enzyme kinetics of one particular thymidine kinase from this library, which contains six amino acid changes at or near the active site, revealed a 35-fold increase in thymidine Kₘ which resulted in reduced competition between prodrug and thymidine at the active site. The mutant is A151V, L159I, I160L, F161A, A168Y and L169F. WO 95/30007, U.S. Pat. No. 5,877,010 and WO 99/19466 (all of which are incorporated herein by reference) describe mutants of HSV-tk which purportedly have increased enzyme activity. For example, WO 99/19466 describes the tk mutants P155A/F161V, P155A/F161V, P155A/D162E, I160L/F161L/A168V/L169M and F161L/A168V/L169Y/L170C. FR 2744731, WO 97/29196 and FR 2751988 relate to mutants of HSV-tk which have mutations in the ATP binding site. WO 95/14102 relates to recombinant adenoviruses which encode tk for use in gene therapy. However, none of these documents describe aberrant splicing of thymidine kinase RNA, or mutations to remove splice sites from tk mRNA.

Methods of using the tk gene in gene therapy are disclosed in WO 90/07936, U.S. Pat. No. 5,837,510, U.S. Pat. No. 5,861,290, WO 98/04290, WO 97/37542 and U.S. Pat. No. 5,631,236, incorporated herein by reference.

A first aspect of the invention provides a polynucleotide encoding a thymidine kinase wherein the thymidine kinase coding region does not contain a functional splice acceptor and/or splice donor site.

As is described in more detail below, splice donor and splice acceptor sites (which may be termed “cryptic” splice donor or splice acceptor sites) may be identified in the natural coding regions of thymidine kinase genes (or cDNAs/mRNAs) and mutations can be engineered that abolish one or more of these sites so that the undesirable splicing does not occur in the target cell.

The polynucleotide may be DNA or RNA and in the context of the invention it will be clear that a reference to a splice acceptor or donor site in a DNA molecule means the part of the DNA molecule (or its complement) which, when transcribed into RNA, contains the given splice acceptor or splice donor sites. Typically the polynucleotide is RNA when present in a retroviral vector (a preferred embodiment of the invention as described below), but it will be appreciated that it may be DNA (for example when the retroviral vector is in a plasmid DNA form or when it is integrated into the genome of the transduced cells), or it may be DNA in other types of vectors which are transcribed into RNA upon introduction into a suitable cell.

The tk coding region of the polynucleotide is not spliced, or able to be spliced, and so a portion of the said coding region is not removed.

Thymidine kinase is a salvage pathway enzyme which phosphorylates natural nucleoside substrates as well as nucleoside analogues (Balaubramaniam et al (1990) J. Gen Virol. 71, 2979-2987). It is useful in gene therapy applications and other applications where it is desirable to selectively destroy a cell because of its ability to phosphorylate relatively non-toxic nucleoside analogues such as acyclovir or ganciclovir creating a toxic product capable of killing the cell expressing thymidine kinase.

The Herpesviridae encode thymidine kinase in their genomes. As with other Herpes simplex virus genes, the HSV-tk gene is a naturally intronless gene (Bordonaro et al (1994) Biochem. Biophys. Res. Comm. 203, 128-132; Otero et al (1998) J. Virol. 72, 9889-9896; Lee et al (1998) J. Cell. Biochem. 69, 104-116). It is preferred if the thymidine kinase is a Herpesviridae thymidine kinase. Representative examples of suitable Herpesviridae thymidine kinase enzymes include herpes simplex virus (HSV) type 1 thymidine kinase, HSV type 2 thymidine kinase, varicella zoster virus thymidine kinase, and the thymidine kinases of marmoset herpesvirus, feline herpesvirus type 1, pseudorabiesvirus, equine herpesvirus type 1, bovine herpesvirus type 1, turkey herpesvirus, Marek's disease virus, herpesvirus saimiri and Epstein Barr virus. It is preferred if the thymidine kinase is from HSV type 1 or HSV type 2.

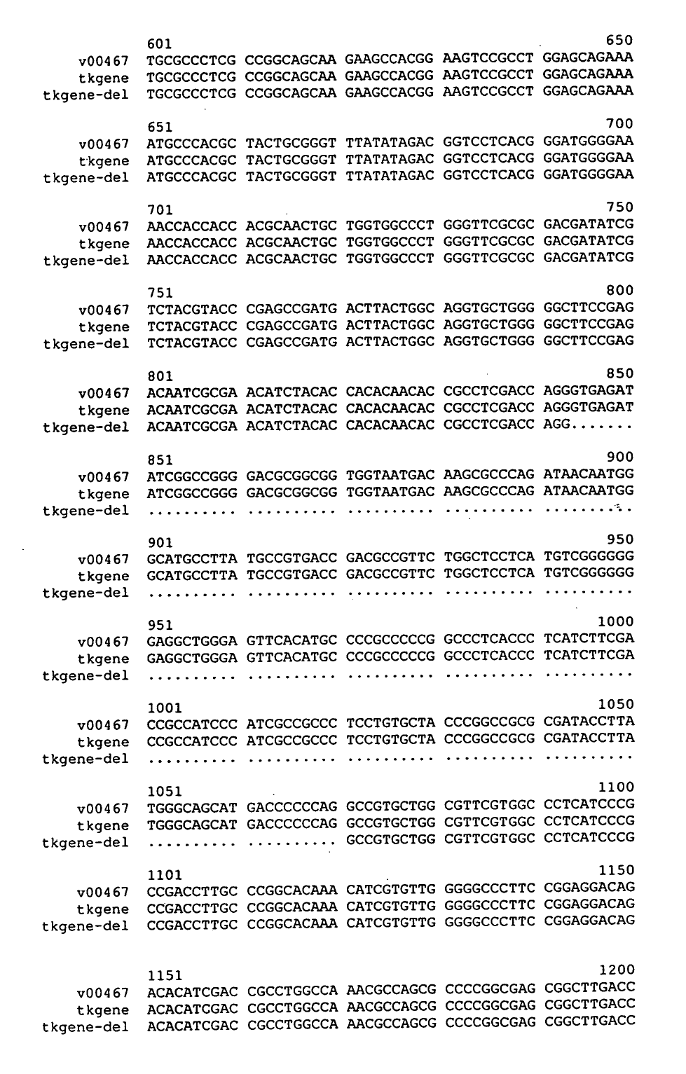

The cDNA sequence of an HSV thymidine kinase is shown in FIG. 11. The complete sequence of the HSV-tk type 1 gene (ATP:thymidine 5′ phosphotransferase, EC 2.7.1.21; accession number V00467, EMBL Database) was described by McKnight S L (Nucleic Acids Res., 77: 244-248; 1980) and Wagner et al (Proc. Natl. Acad. Sci. 78(3) 1441-45; 1980). According to this numbering scheme; the HSV-tk gene sequence used in the retroviral vector construct in Example 1 is located at positions 516 for the 5′ end and 1646 for the 3′ end. The deleted fragment of the HSV-tk gene was located between positions 844 and 1071 for the 5′ and 3′ ends, respectively (Example 1, FIG. 11).

A splice donor site is a site in RNA which lies at the 5′ side of the RNA which is removed during the splicing process and which contains the site which is cut and rejoined to a nucleotide residue within a splice acceptor site. Thus, a splice donor site is the junction between the end of an intron, typically terminating in the dinucleotide AG, and the start of the next exon.

A splice acceptor site is a site in RNA which lies at the 3′ side of the RNA which is removed during the splicing process and which contains the site which is cut and rejoined to a nucleotide residue within a splice donor site. Thus, a splice acceptor site is the junction between the end of an exon and the start of the downstream intron, typically commencing with the dinucleotide GT.

The portion of RNA which is removed (or “spliced out”) during splicing is typically called an intron, and the two pieces of RNA either side of the intron that are joined by splicing are typically called exons.

Although “consensus” sequences have been produced which may be used to describe, and possibly identify, splice sites, it is well known that “cryptic” splice sites occur which may not conform to the consensus but nevertheless function as splice sites. For the avoidance of doubt, in the context of the invention the splice sites referred to include cryptic splice sites. Thus, in particular, the polynucleotides of the invention do not contain a cryptic splice donor site and/or a cryptic splice acceptor site. A cryptic splice site is a sequence which resembles an authentic splice junction site and which can, under some circumstances, participate in an RNA splicing reaction.

By “does not contain a functional splice acceptor site” we include the meaning that the coding region in the polynucleotide (or as the case may be, expression vector) does not contain a portion of RNA (or DNA which can be transcribed into RNA or its complement) which can serve as a splice acceptor site in combination with a splice donor site present in the polynucleotide or expression vector.

By “does not contain a functional splice donor site” we include the meaning that the coding region in the polynucleotide (or as the case may be, expression vector) does not contain a portion of RNA (or DNA which can be transcribed into RNA or its complement) which can serve as a splice donor site in combination with a splice acceptor site present in the polynucleotide or expression vector.

The polynucleotide of the invention may contain a splice acceptor site in the coding region of tk but if it does, it does not contain a splice donor site, and no splicing out of a portion of the coding region occurs.

The polynucleotide may contain a splice donor site in the coding region of tk but if it does, it does not contain a splice acceptor site, and no splicing out of a portion of the coding region occurs. Preferably, the polynucleotide does not contain within the coding region of tk a splice acceptor site and does not contain a splice donor site.

Coding regions encoding thymidine kinase can readily be made which do not contain a functional splice acceptor site and/or a splice donor site using standard mutagenesis techniques such as oligonucleotide-directed mutagenesis or polymerase chain reaction based methods.

Oligonucleotide site-directed mutagenesis in essence involves hybridizing an oligonucleotide coding for a desired mutation with a single strand of DNA containing the region to be mutated and using the single strand as a template for extension of the oligonucleotide to produce a strand containing the mutation. This technique, in various forms, is described in Zoller and Smith (1982) Nucl. Acids Res. 10, 6487.

Polymerase chain reaction (PCR) in essence involves exponentially amplifying DNA in vitro using sequence specific oligonucleotides. The oligonucleotides can incorporate sequence alterations if desired. The polymerase chain reaction technique is described in Mullis and Fuloona (1987) Meth. Enz. 155, 335. Examples of mutagenesis using PCR are described in Ho et al (1989) Gene 77, 51.

The presence of splice sites in a coding region of a thymidine kinase coding sequence, can be determined, for example using the methods described in Example 1. As described in more detail with respect to tk in Example 1, by subcloning of the transduced bulk populations and characterisation of the derived subclones by molecular techniques, such as sequence analysis, it is possible to identify consensus sequences with high likelihood of being recognised by the splicing machinery of the virus producer cells (the so called cryptic splice sites). These consensus sequences, in particular, those directly located in the coding region or regulatory elements of the transgenes, can be avoided when designing new vector constructs by changing one or more of the splice sites as herein described.

Once the splice site or splice sites have been identified, it is possible to modify its/their sequence so that the splice site or sites is/are no longer functional.

Mutation of splice sites can readily be shown to abolish undesirable splicing. As illustrated in Example 1, those subclones carrying the deleted HSV-tk gene showed inadequate expression of the gene when the cells were exposed to GCV. The cell proliferation, assessed by the incorporation of ³H thymidine, was similar to that observed in the control non-transduced cells. The characterisation of the molecular mechanism for the resistance to GCV allowed us to develop a PCR to amplify specifically the deleted HSV-tk gene in the transduced bulk populations. Primary human T-cells as well as CEM, a normal human T-cell line, were transduced using a vector carrying the splice-corrected HSV-tk gene (ie a gene in which the splice sites had been removed by mutagenesis). The PCR analysis performed on these populations failed to amplify the expected band of the truncated HSV-tk gene. These transduced bulk populations have been subcloned in the same manner as was done for the non-modified HSV-tk gene. Any of the clones with the non-modified HSV-tk gene analysed showed the expected deletion at positions 842 and 1070 of the HSV-tk sequence.

Therefore, a further aspect of the invention provides a method of making a polynucleotide according to the first aspect of the invention, the method comprising (1) determining whether the thymidine kinase coding region contains a functional splice acceptor and/or splice donor site and (2) if it does, mutating at least one of the splice acceptor and/or splice donor sites to make them non-functional. Typically, step (1) comprises analysing mRNA transcribed from at least part of the natural coding region to determine whether the mRNA indicates that a splicing event has occurred using a splice site within the coding region. This may conveniently be carried out using PCR methods as herein described. Typically, the mutations in step (2) are introduced using site-directed mutagenesis, for example by using mismatched oligonucleotides.

In a preferred embodiment of the invention, the splice site(s) is modified taking note of the genetic code such that a codon is changed to a degenerate codon which codes for the same amino acid residue. In this way, it is possible to make coding regions for the protein of interest which encode wild type protein but which do not contain a functional splice acceptor and/or splice donor site.

In a further preferred embodiment of the invention, the thymidine kinase coding region is modified so that it encodes an enzyme, which compared to the wild type, contains mutations that enhance the enzymatic activity.

Mutants of thymidine kinase are described in, for example, Kokoris et al (1999) Gene Therapy 6, 1415-1426, WO 95/30007, U.S. Pat. No. 5,877,010, WO 99/19466, FR 2744731, WO 97/29196 and FR 2751988, all of which are incorporated herein by reference. Thus, the invention includes polynucleotides and expression vectors encoding a thymidine kinase wherein the thymidine kinase coding region does not contain a functional splice acceptor site and/or splice donor site and which encodes a thymidine kinase which, when compared to wild type, contains mutations that enhance the enzymatic activity. Particularly preferred mutations are those described in the above-mentioned journal article and patent applications and patents.

It will be appreciated that the polynucleotide of the invention may contain only a coding region for the thymidine kinase. However, it is preferred if the polynucleotide further comprises, in operable linkage, a portion of nucleic acid that allows for efficient translation of the coding sequence in the target cell. It is further preferred if the polynucleotide (when in a DNA form) further comprises a promoter in operable linkage which allows for the transcription of the coding region and the portion of nucleic acid that allows for efficient translation of the coding region in the target cell. A promoter is an expression control element formed by a DNA sequence that permits binding of RNA polymerase and transcription to occur.

The polynucleotide may be used in accordance with known techniques, appropriately modified in view of the teachings contained herein, to construct an expression vector, which is then used to transform an appropriate host or target cell for the expression and production of thymidine kinase. Such techniques include those disclosed in U.S. Pat. No. 4,440,859 issued 3 Apr. 1984 to Rutter et al, U.S. Pat. No. 4,530,901 issued 23 Jul. 1985 to Weissman, U.S. Pat. No. 4,582,800 issued 15 Apr. 1986 to Crowl, U.S. Pat. No. 4,677,063 issued 30 Jun. 1987 to Mark et al, U.S. Pat. No. 4,678,751 issued 7 Jul. 1987 to Goeddel, U.S. Pat. No. 4,704,362 issued 3 Nov. 1987 to Itakura et al, U.S. Pat. No. 4,710,463 issued 1 Dec. 1987 to Murray, U.S. Pat. No. 4,757,006 issued 12 Jul. 1988 to Toole, Jr. et al, U.S. Pat. No. 4,766,075 issued 23 Aug. 1988 to Goeddel et al and U.S. Pat. No. 4,810,648 issued 7 Mar. 1989 to Stalker, all of which are incorporated herein by reference.

The polynucleotide (typically in the DNA form) may be joined to a wide variety of other DNA sequences for introduction into an appropriate host. The companion DNA will depend upon the nature of the host, the manner of the introduction of the DNA into the host, and whether episomal maintenance or integration is desired.

Generally, the polynucleotide (DNA) is inserted into an expression vector, such as a retroviral vector plasmid, in the proper orientation and correct reading frame for expression. If necessary, the DNA may be linked to the appropriate transcriptional and translational regulatory control nucleotide sequences recognised by the desired host, although such controls are generally available in the expression vector. The vector is then introduced into the host (or target cell) through standard techniques. Generally, not all of the hosts or target cells will be transformed by the vector. Therefore, it will be necessary to select for transformed host or target cells. One selection technique involves incorporating into the expression vector a DNA sequence, with any necessary control elements, that codes for a selectable trait in the transformed (or transduced) cell, such as antibiotic resistance. Alternatively, the gene for such a selectable trait can be on another vector, which is used to co-transform (or co-transduce) the desired host cell. When the protein of interest is tk, the presence of tk may be detected in a transformed cell, for example by measuring tk enzyme activity, or by detecting tk expression immunologically. The expression of the HSV-tk gene may conveniently be determined by measuring the inhibition of cell proliferation assessed by the incorporation of ³H-thymidine into the newly synthesised DNA.

Thus, a second aspect of the invention provides an expression vector comprising a polynucleotide of the first aspect of the invention. The expression vector is a vector which allows for the tk to be taken up by the target cell and for the coding region to be transcribed and translated. In the context of the invention, the target cell is typically a cell which can undertake splicing. Preferably the cell is a mammalian cell and more preferably it is a human cell. Thus, a preferred embodiment of the invention is an expression vector which allows for the efficient expression of the tk, in a mammalian cell, more particularly in a human cell.

The vector of the invention is suitably one which has been adapted for use in gene therapy. Typically, the vector is one which allows for expression in a mammalian cell, preferably a human cell. In particular, it is preferred if the vector is one which can selectively target cells which it is desired to destroy. It is further preferred if the vector is one which allows for selective expression in the target cell by using promoter sequences which work selectively in the target cell type. These embodiments are described in more detail below.

The expression vector is conveniently a viral vector; more particularly it is preferred that the vector is a retroviral vector. However, the polynucleotides, expression vectors and methods of the invention include those expression vectors in which the splicing machinery of the virus producer cells might interfere during synthesis of the infectious virus particles, such as retroviruses, adenoviruses, lentiviruses other such viruses known in the art. Thus, the invention relates to any expression vector in which the vector-derived pre-mRNA can be recognised and subsequently processed by the splicing machinery of the host cells leading to an inadequate expression of the transgene (ie protein of interest).

Polynucleotides and expression vectors of the invention may be made by any suitable method. For example, a variety of methods have been developed to operably link DNA to vectors via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion as described earlier, is treated with bacteriophage T4 DNA polymerase or E. coli DNA polymerase 1, enzymes that remove protruding, 3′-single-stranded termini with their 3′-5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of sources including International Biotechnologies Inc, New Haven, Conn., USA.

A desirable way to modify the DNA encoding the polypeptide of the invention is to use the polymerase chain reaction as disclosed by Saiki et al (1988) Science 239, 487-491.

In this method the DNA to be enzymatically amplified is flanked by two specific oligonucleotide primers which themselves become incorporated into the amplified DNA. The said specific primers may contain restriction endonuclease recognition sites which can be used for cloning into expression vectors using methods known in the art.

It will be appreciated that the polynucleotide of the invention or the expression vector of the invention may readily be made using molecular biological techniques which are well known in the art, such as those described in Sambrook et al (1989). Molecular cloning, a laboratory manual, 2^{nd} edition, Cold Spring Harbor Press, Cold Spring Harbor, N.Y.

A third aspect of the invention provides a host cell comprising a polynucleotide of the first aspect of the invention or an expression vector of the second aspect of the invention.

The host cell may be a cell used for propagating the polynucleotide or expression vector so that sufficient quantities of it may be made for further use. For example, the host cell may be a bacterial cell (such as E. coli) which is used to produce DNA. Thus, for example, plasmid DNA forms of retroviral vectors may be produced in E. coli.

The host cell may be a cell for packaging and propagating a virus, such as retroviral packaging cell lines which are well known in the art.

The host cell may be a cell in an animal or patient (whether human or animal) which it is desired to destroy. As is discussed in more detail below, the polynucleotide and vector of the invention are useful to target to cells to be destroyed, and for the cells (which express tk under certain conditions) to be contacted with an agent which is substantially non-toxic which is converted to a toxic form by tk.

Host cells that have been transformed by the recombinant DNA or RNA of the invention may be made using methods well known in the art.

A fourth aspect of the invention provides a pharmaceutical composition comprising a polynucleotide of the first aspect of the invention or air expression vector of the second aspect of the invention further comprising a pharmaceutically acceptable carrier.

The pharmaceutically acceptable carrier is selected according to the physical and biological form of the polynucleotide or expression vector of the invention so that it is compatible. Typically, it is sterile and pyrogen free. When the vector is a viral vector, typically the pharmaceutical composition may include some agents which stabilise the virus, such as a low concentration of a non-ionic detergent, or such as a protein (eg serum albumin). The formulations may conveniently be presented in unit dosage form and may be prepared by any of the methods well known in the art of gene therapy and pharmacy. Such methods include the step of bringing into association the polynucleotide or expression vector with the carrier which constitutes one or more accessary ingredients.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

A fifth aspect of the invention provides a polynucleotide of the first aspect of the invention or an expression vector of the second aspect of the invention for use in medicine. That is to say, the polynucleotide or expression vector are packaged and presented for use in medicine.

The polypeptides and expression vectors of the invention are useful in treating a patient, particularly a human patient, who has a target cell to be destroyed.

A sixth aspect of the invention provides a method of destroying cells the method comprising introducing into the cells a polynucleotide according to the first aspect of the invention or an expression vector according to the second aspect of the invention, allowing the cells to express thymidine kinase, and contacting the cells with a substantially non-toxic agent which is converted by thymidine kinase to a toxic agent. The introduction into the cells of the polynucleotide or expression vector, and the contacting of the cells with the substantially non-toxic agent, may be in any order.

The cells to be destroyed may be cells in vitro, such as cells which are being grown in culture, or they may be cells which are part of an animal, including a human. It may be desirable to destroy the cells for a variety of reasons. For example, it may be desirable to destroy the cell because it is, or has the potential of becoming, a cancer cell. In a further embodiment, it may be desirable to destroy the cell because it is a progenitor cell for a line of cells which it is desired not to produce. For example, the cell may be a stem cell in an animal which is being used in an experimental system. The stem cell may be destroyed in which case the cell lines derived from the stem cell will not be produced.

The polynucleotide or expression vector is placed in contact with the cells so that it is taken up by the target cells. Once in the cells the polynucleotide or genetic material from the expression vector may stably integrate into the cell's genome or it may be maintained episomally. Either way, and in any event, the cells express thymidine kinase (although for some systems this may be dependent upon the cells being subjected to a stimulus, see below). In order for the target cells (which are expressing the thymidine kinase) to be killed they are contacted with a substantially non-toxic agent which is converted by thymidine kinase into a toxic agent.

A seventh aspect of the invention provides a method of treating a patient with cells in need of destruction the method comprising introducing into the patient a polynucleotide according to the first aspect of the invention or an expression vector according to the second aspect of the invention, allowing the polynucleotide or expression vector to be taken up by the cells, allowing the cells to express thymidine kinase, and administering to the patient a substantially non-toxic agent which is converted by thymidine kinase to a toxic agent. The substantially non-toxic agent may be administered before, during or after the introduction of the polynucleotide or expression vector.

Preferably, the genetic construct (by which we mean the polynucleotide or expression vector of the invention) is adapted for delivery to a cell, preferably a human cell. More preferably, the genetic construct is adapted for delivery to a cell in an animal body, more preferably a mammalian body; most preferably it is adapted for delivery to a cell in a human body.

Means and methods of introducing a genetic construct into a cell in an animal body are known in the art. For example, the constructs of the invention may be introduced into the target cells by any convenient method, for example methods involving retroviruses, so that the construct is inserted into the genome of the tumour cell. For example, in Kuriyama et at (1991) Cell Struc. and Func. 16, 503-510 purified retroviruses were administered. Retroviruses provide a potential means of selectively infecting cancer cells because they can only integrate into the genome of dividing cells; most normal cells surrounding cancers are in a quiescent, non-receptive stage of cell growth or may be dividing less rapidly than the tumour cells. Retroviral DNA constructs which contain a suitable promoter segment and a polynucleotide encoding thymidine kinase as described may be made using methods well known in the art. To produce active retrovirus from such a construct it is usual to use an amphotrophic packaging cell line. Transfection of the cell line is conveniently by calcium phosphate co-precipitation, and stable transformants are selected by addition of G418 to a final concentration of 1 mg/ml (assuming the retroviral construct contains a neo^{R} gene). Independent transduced colonies are isolated, may be selected and expanded and the culture supernatant removed, filtered through a 0.45 μm pore-size filter and stored at −70°. For the introduction of the retrovirus into the tumour cells in vitro, it is convenient to incubate the retroviral supernatant to which 10 μg/ml Polybrene has been added with the tumour cells. For introduction of retroviruses into a tumour in situ it is usual for the retroviral supernatant to be injected into the area of the tumour. For tumours exceeding 10 mm in diameter it is appropriate to inject between 0.1 ml and 1 ml of retroviral supernatant of an appropriate titre; preferably 0.5 ml.

Alternatively, as described in Culver et al (1992) Science 256, 1550-1552, cells which produce retroviruses are injected into the site of the target cells, such as a tumour. The retrovirus-producing cells so introduced are engineered to actively produce retroviral vector particles so that continuous productions of the vector occurred within the tumour mass in situ. Thus, proliferating target cells such as tumour cells can be successfully transduced in vivo if mixed with retroviral vector-producing cells. Thus, by “introducing into the patient a polynucleotide according to the first aspect of the invention or an expression vector according to the second aspect of the invention” we include introducing into the patient retrovirus-producing cells as described.

Targeted retroviruses are also available for use in the invention; for example, sequences conferring specific binding affinities may be engineered into preexisting viral env genes (see Miller & Vile (1995) Faseb J. 9, 190-199 for a review of this and other targeted vectors for gene therapy). The tropism of a retroviral vector can be altered by the incorporation of foreign or hybrid envelope proteins (Battini J L, et al J. Virol 66: 1468-1475; 1992). This can be achieved by insertion of monoclonal antibodies to mouse ecotropic retroviral particles. Alternatively, any chemical modification such as lactose binding to virus particles can increase the range possible target cells for transduction or confer a predictably altered recognition specificity. Retrovirus particles displaying non-viral polypeptides may be used for specific target cells through the non-viral moiety.

Other methods involve simple delivery of the genetic construct into the cell for expression therein either for a limited time or, following integration into the genome, for a longer time. An example of the latter approach includes (preferably tumour-cell-targeted) liposomes (Nässander et al (1992) Cancer Res. 52, 646-653).

Immunoliposomes (antibody-directed liposomes) are especially useful in targeting to cancer cell types which over-express a cell surface protein for which antibodies are available (see Table for examples). For the preparation of immuno-liposomes MPB-PE (N-[4-(p-maleimidophenyl)butyryl]-phosphatidylethanolamine) is synthesised according to the method of Martin & Papahadjopoulos (1982) J. Biol. Chem. 257, 286-288. MPB-PE is incorporated into the liposomal bilayers to allow a covalent coupling of the antibody, or fragment thereof, to the liposomal surface. The liposome is conveniently loaded with the DNA or other genetic construct of the invention for delivery to the target cells, for example, by forming the said liposomes in a solution of the DNA or other genetic construct, followed by sequential extrusion through polycarbonate membrane filters with 0.6 μm and 0.2 μm pore size under nitrogen pressures up to 0.8 MPa. After extrusion, entrapped DNA construct is separated from free DNA construct by ultracentrifugation at 80 000×g for 45 min. Freshly prepared MPB-PE-liposomes in deoxygenated buffer are mixed with freshly prepared antibody (or fragment thereof) and the coupling reactions are carried out in a nitrogen atmosphere at 4° C. under constant end over end rotation overnight. The immunoliposomes are separated from unconjugated antibodies by ultracentrifugation at 80 000×g for 45 min. Immunoliposomes may be injected intraperitoneally or directly into the tumour.

Although immunoliposomes may be used, it is also possible to use liposomes which target cells by virtue of containing a peptide moiety which can bind to a target cell. Such peptide moieties include ligands for receptors that may be selectively expressed (or overexpressed) on the target cell.

The DNA may also be delivered by adenovirus wherein it is present within the adenovirus particle, for example, as described below.

Other methods of delivery include adenoviruses carrying external DNA via an antibody-polylysine bridge (see Curiel Prog. Med. Virol. 40, 1-18) and transferrin-polycation conjugates as carriers (Wagner et al (1990) Proc. Natl. Acad. Sci. USA 87, 3410-3414). In the first of these methods a polycation-antibody complex is formed with the DNA construct or other genetic construct of the invention, wherein the antibody is specific for either wild-type adenovirus or a variant adenovirus in which a new epitope has been introduced which binds the antibody. The polycation moiety binds the DNA via electrostatic interactions with the phosphate backbone. It is preferred if the polycation is polylysine.

In the second of these methods, a high-efficiency nucleic acid delivery system that uses receptor-mediated endocytosis to carry DNA macromolecules into cells is employed. This is accomplished by conjugating the iron-transport protein transferrin to polycations that bind nucleic acids. Human transferrin, or the chicken homologue conalbumin, or combinations thereof are covalently linked to the small DNA-binding protein protamine or to polylysines of various sizes through a disulfide linkage. These modified transferrin molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilobase pairs). When complexes of transferrin-polycation and the DNA constructs or other genetic constructs of the invention are supplied to the tumour cells, a high level of expression from the construct in the cells is expected.

High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. This approach appears to rely on the fact that adenoviruses are adapted to allow release of their DNA from an endosome without passage through the lysosome, and in the presence of, for example transferrin linked to the DNA construct or other genetic construct of the invention, the construct is taken up by the cell by the same route as the adenovirus particle.

This approach has the advantages that there is no need to use complex retroviral constructs; there is no permanent modification of the genome as occurs with retroviral infection; and the targeted expression system is coupled with a targeted delivery system, thus reducing toxicity to other cell types.

When the target cells are in a tumour, it may be desirable to locally perfuse a tumour with the suitable delivery vehicle comprising the genetic construct for a period of time; additionally or alternatively the delivery vehicle or genetic construct can be injected directly into accessible tumours.

Alternative targeted delivery systems are also known such as the modified adenovirus system described in WO 94/10323 wherein, typically, the DNA is carried within the adenovirus, or adenovirus-like, particle. Michael et al (1995) Gene Therapy 2, 660-668 describes modification of adenovirus to add a cell-selective moiety into a fibre protein Mutant adenoviruses which replicate selectively in p53-deficient human tumour cells, such as those described in Bischoff et al (1996) Science 274, 373-376 are also useful for delivering the genetic construct of the invention to a cell. Thus, it will be appreciated that a further aspect of the invention provides a virus or virus-like particle comprising a genetic construct of the invention. Other suitable viruses or virus-like particles include HSV, AAV, vaccinia and parvovirus.

It will be appreciated that in the first and second aspects of the invention the polynucleotide or expression vector need not be one which has a target cell-selective promoter to drive the expression of thymidine kinase, but it is preferred if it is in order to give selectivity.

Preferably the target cell-selective promoter is a tumour cell-selective promoter when the polynucleotides, expression vectors and methods of the invention are used to treat tumours. In addition, other types of target cell-selective promoters may be useful in other applications.

Preferably, the target cell-selective promoter is a cell-selective promoter when the polynucleotides, expression vectors and methods of the invention are used to ensure that the therapeutic gene product is only made in the desired target cells. This can be achieved by limiting gene expression with the use of transcriptional control elements or tissue-specific promoters. Gene expression can be regulated by the availability (eg presence or absence) of transcription factors that recognise specific regulatory elements present in the promoter region of the genes.

Regulatory elements that confer tissue-specific expression can be included in viral vectors either in the body of the vector or in addition to, or in place of, the viral promoter or regulatory elements. Tissue-specific gene expression may be required in transduction of haemopoietic stem cells where the goal is to express the therapeutic gene exclusively in a differentiated cell lineage (eg erythroid, T-cell or macrophages) (Grande-A et al Blood 1999, 15; 93: 3276-85). There are a number of promoters available which have been isolated from genes specifically or preferentially expressed in particular tissues (Huber et al 1991; Proc. Natl. Sci. USA 88: 8039-43; Hafenrichter et al, 1994; Blood 84: 3394-3404).

Depending on the site of integration of the expression vector in the host-cell genome, the tissue specific regulation conferred upon the promoter may be overridden by strong cellular regulatory elements located in the vicinity of the integration site. DNA sequences, termed locus control region (LCR), can be used to confer position independent and high-level expression of the transgenes (Dillon and Groveld, 1993; Trends Genet. 9: 134-7).

An alternative strategy to ensure position-independent expression of genes in expression vectors is to shield them from the effect of enhancers or repressors located in the vicinity of the integration site (Duch et al, 1994; J. Virol. 68: 5596-5601). A number of such insulators have been identified in mammalian and non-mammalian cells and these could be incorporated into future vector designs (Kalos and Fournier, 1995; Mol. Cell Biol. 15: 198-207; Roseman et al, 1995; Development, 121: 3573-3582).

Useful genetic elements which are target cell-selective promoters are given below but new ones are being discovered all of the time which will be useful in this embodiment of the invention.

The tyrosinase and TRP-1 genes both encode proteins which play key roles in the synthesis of the pigment melanin, a specific product of melanocytic cells. The 5′ ends of the tyrosinase and tyrosinase-related protein (TRP-1) genes confer tissue specificity of expression on genes cloned downstream of these promoter elements.

The 5′ sequences of these genes are described in Bradl, M. et al (1991) Proc. Natl. Acad. Sci. USA 88, 164168 and Jackson, I. J. et al (1991) Nucleic Acids Res. 19, 3799-3804.

Prostate-specific antigen (PSA) is one of the major protein constituents of the human prostate secretion. It has become a useful marker for the detection and monitoring of prostate cancer. The gene encoding PSA and its promoter region which directs the prostate-specific expression of PSA have been described (Lundwall (1989) Biochem. Biophys. Res. Comm. 161, 1151-1159; Riegman et al (1989) Biochem. Biophys. Res. Comm. 159, 95-102; Brawer (1991) Acta Oncol. 30, 161-168).

Carcinoembryonic antigen (CEA) is a widely used tumour marker, especially in the surveillance of colonic cancer patients. Although CEA is also present in some normal tissues, it is apparently expressed at higher levels in tumorous tissues than in corresponding normal tissues. The complete gene encoding CEA has been cloned and its promoter region analysed. A CEA gene promoter construct, containing approximately 400 nucleotides upstream from the translational start, showed nine times higher activity in the adenocarcinoma cell line SW303, compared with the HeLa cell line. This indicates that cis-acting sequences which convey cell type specific expression are contained within this region (Schrewe et al (1990) Mol. Cell. Biol. 10, 2738-2748).

The mucin gene, MUC1, contains 5′ flanking sequences which are able to direct expression selectively in breast and pancreatic cell lines, but not in non-epithelial cell lines as taught in WO 91/09867.

The alpha-fetoprotein (AFP) enhancer may be useful to drive pancreatic tumour-selective expression (Su et al (1996) Hum. Gene Ther. 7, 463-470).

It will be appreciated that it may be desirable to be able to temporally regulate expression of the said thymidine kinase in the cell. This is particularly the case when the polynucleotide or expression vector encoding thymidine kinase is introduced into a cell within the body of a patient or animal. Thus, it may be desirable that expression of the said thymidine kinase is directly or indirectly under the control of a promoter that may be regulated, for example by the concentration of a small molecule that may be administered to the animal or patient when it is desired to activate or repress (depending upon whether the small molecule effects activation or repression of the said promoter) expression of the said thymidine kinase. It will be appreciated that this may be of particular benefit if the expression construct is stable, ie capable of expressing the said thymidine kinase in the said cell for a period of at least one week, one, two, three, four, five, six, eight months or one or more years. A preferred construct of the invention may comprise a regulatable promoter. Examples of regulatable promoters include those referred to in the following papers: Rivera et al (1999) Proc Natl Acad Sci USA 96(15), 8657-62 (control by rapamycin, an orally bioavailable drug, using two separate adenovirus or adeno-associated virus (AAV) vectors, one encoding an inducible human growth hormone (hGH) target gene, and the other a bipartite rapamycin-regulated transcription factor); Magari et al (1997) J Clin Invest 100(11), 2865-72 (control by rapamycin); Bueler (1999) Biol Chem 380(6), 613-22 (review of adeno-associated viral vectors); Bohl et al (1998) Blood 92(5), 1512-7 (control by doxycycline in adeno-associated vector); Abruzzese et al (1996) J Mol Med 74(7), 379-92 (reviews induction factors, eg hormones, growth factors, cytokines, cytostatics, irradiation, heat shock and associated responsive elements).

The polynucleotide or expression vector is introduced into the patient to be treated in any suitable way. Sufficient time is allowed for the target cell to receive the polynucleotide or expression vector and for it to be taken up by the cell and to express thymidine kinase. The patient is then administered a sufficient quantity of non-toxic agent which is converted by thymidine kinase into a toxic agent for the non-toxic agent to come into contact with and enter the target cell expressing thymidine kinase, and for it to be converted by the enzyme into an amount of toxic agent sufficient to kill the target cell.

The substantially non-toxic agent which is converted by thymidine kinase into a toxic agent may be any one of ganciclovir, acyclovir, trifluorothymidine, 1-[2-deoxy,2-fluoro,β-D-arabino furanosyl]-5-iodouracil, ara-A, ara 1,1-β-D arabino furanosyl thymine, 5-ethyl-2′-deoxyuridine, 5-iodo-5′-amino-2,5′-dideoxyuridine, idoxuridine, AZT, AIV, dideoxycytidine and Ara C. Bromovinyl deoxyuridine (BVDU) may also be used. Suitably, any nucleoside analogue or non-related chemical compound susceptible of being metabolised by the tk leading to the killing of the engineered cells as well as those cells exposed to the metabolic product of the prodrug used. Preferably, the substantially non-toxic agent is ganciclovir. Ganciclovir is (9-{[2-hydroxy-1-(hydroxymethyl)ethoxyl methyl} guanosine). Acyclovir is (9-[2-hydroxyethoxy) methyl]guanosine). AraA is (adenosine arabinoside, vivarabine). AZT is 3′ aziod-3′ thymidine. AIU is 5-iodo-5′ amino 2′,5′-dideoxyuridine. AraC is cytidine arabinoside.

An eighth aspect of the invention provides a method of treating a patient with cells in need of destruction, the method comprising (1) removing the cells from the patient or donor of cells, (2) introducing into the cells ex vivo a polynucleotide according to the first aspect of the invention or an expression vector according to the second aspect of the invention, (3) introducing the modified cells into the patient which may or may not be expressing thymidine kinase when so introduced, (4) optionally, allowing the cells to express thymidine kinase if not so expressing and (5) administering to the patient a substantially non-toxic agent which is converted by thymidine kinase into a toxic agent.

In relation to step (1), the target cells can be obtained from different sources depending on the therapeutic strategy desired. In cancer gene therapy, the remission of the tumours will involve cells derived from the patient. In allogeneic bone marrow transplantation, cells will be obtained from donors to be transplanted into an adequate recipient. The in vivo administration of the polynucleotide, or expression vector into the tumour mass could be a feasible alternative to the in vitro engineering of the cells.

A ninth aspect of the invention provides the use of a polynucleotide according to the first aspect of the invention or an expression vector according to the second aspect of the invention in the manufacture of a medicament for destroying cells in a patient wherein the patient has been, is being, or will be administered a substantially non-toxic agent which is converted by thymidine kinase to a toxic agent.

A tenth aspect of the invention provides the use of a substantially non-toxic agent which is converted by thymidine kinase to a toxic agent in the manufacture of a medicament for destroying cells in a patient wherein the patient has been, is being, or will be administered with a polynucleotide according to the first aspect of the invention or an expression vector according to the second aspect of the invention.

An eleventh aspect of the invention provides a therapeutic system (or it may be termed a “kit of parts”) comprising a polynucleotide according to the first aspect of the invention or an expression vector according to the second aspect of the invention and a substantially non-toxic agent which is converted by thymidine kinase to a toxic agent. The substantially non-toxic agent may be any of the aforementioned agents that are converted into a toxic form by the action of thymidine kinase; preferably the non-toxic agent is ganciclovir.

The invention will now be described in more detail, for the purposes of illustration only, in the following Examples and Figures wherein:

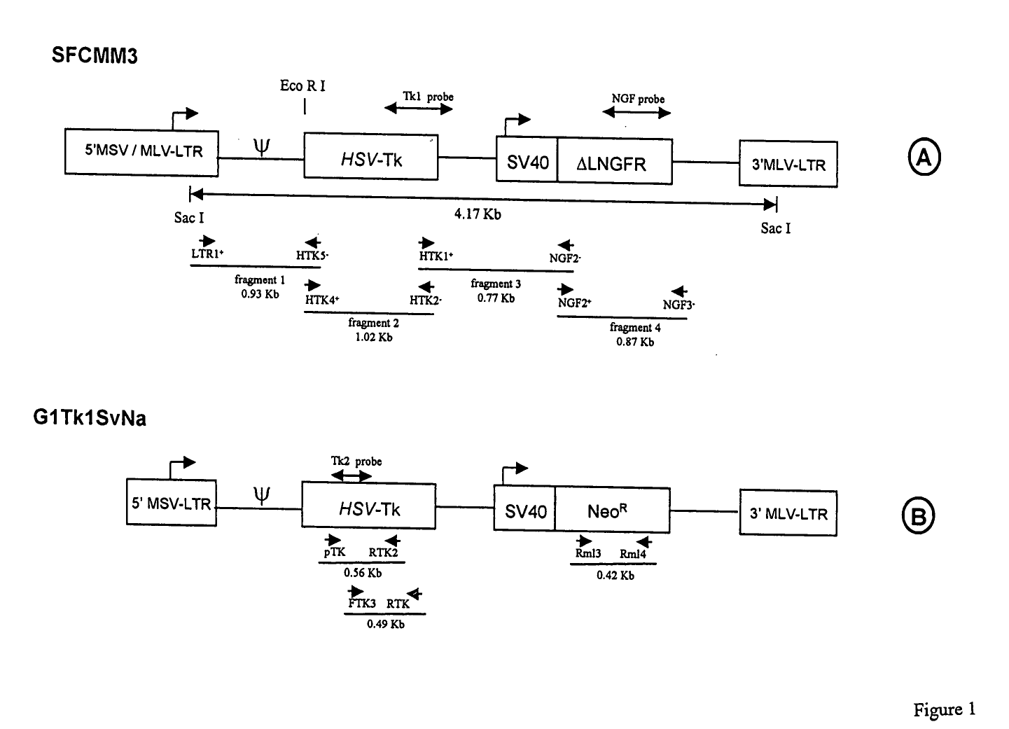

FIG. 1 shows a schematic representation of the proviral forms of SFCMM3 (A) and G1Tk1SvNa (13) vectors used for the transduction of CEM cells and primary T-lymphocytes. LTR, long terminal repeats derived from Moloney murine leukaemia virus (MLV) and Moloney sarcoma virus (MSV). HSV-Tk, herpes simplex virus gene sequence. SV40, simian virus 40 early promoter. ΔLNGFR, low-affinity nerve growth factor receptor cDNA truncated in the cytoplasmic domain. Neo^{R}: neomycin phosphotransferase cDNA. Restriction enzyme sites for EcoRI and SacI used for the digestion of genomic DNAs derived from the CEM-Tk sub-clones are indicated. The Tk1 and NGF probes were used to identify the provirus sequences in the genomic DNA. The Tk2 probe was used also to confirm the specificity of PCR products. The location of the PCR primers designed to amplify the SFCMM3 and G1Tk1SvNa provirus sequences are shown together with the sizes of PCR products.

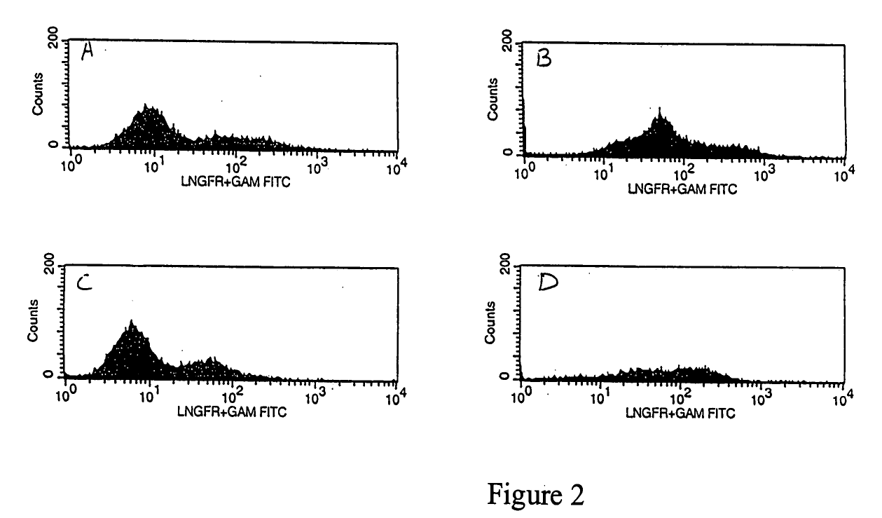

FIG. 2 shows the detection of ΔLNGFR expression by FACS analysis in retrovirally transduced T-cell lines. CEM and Jurkat cells (human cell lines) transduced unselected populations (A and C, respectively). Enrichment of ΔLNGFRexpressing CEM and Jurkat cells after one round of positive immunomagnetic selection with magnetic beads (B and D, respectively). Results shown correspond to one representative experiment.

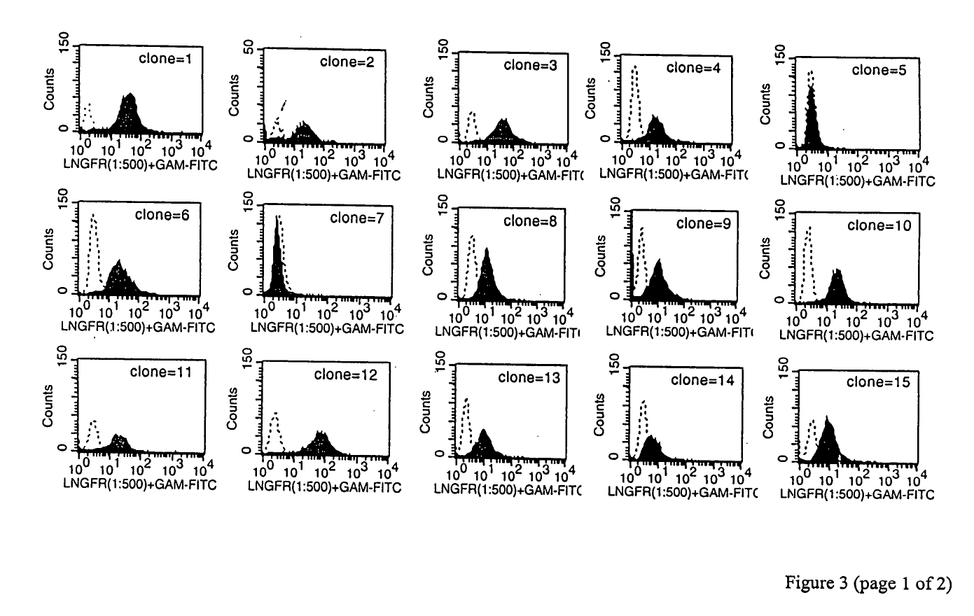

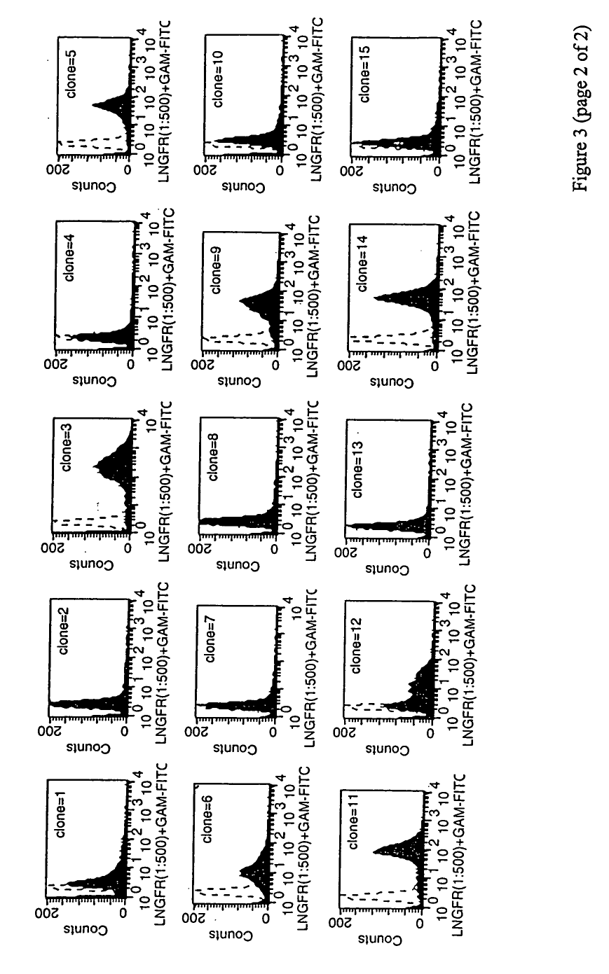

FIG. 3 shows the expression by FACS analysis of the ΔLNGFR transgene in the TK-CEM (A) and TK-Jurkat (B) derived sub-clones. Cells were incubated with an unconjugated mouse anti-human LNGFR MoAb and subsequently stained with a FITC labelled goat anti-mouse MoAb.

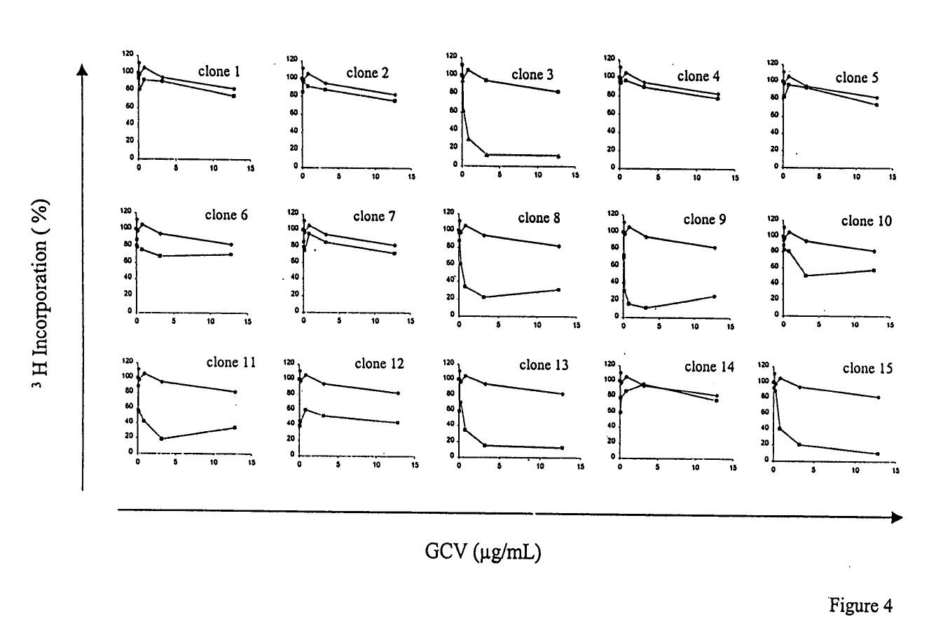

FIG. 4 shows a GCV-induced cytotoxic assay for the determination of the HSV-tk transgene in the TK-CEM derived sub-clones. Cells were incubated for 4 days in the presence of increasing concentration of GCV (range from 0.05 to 12.5 μg/mL). Cell proliferation was measured by the incorporation of ³H— thymidine into the cell DNA. Results are expressed as percentage of incorporated ³H-thymidine at each GCV concentration respect to the incorporation obtained in the absence of GCV. (♦) non-transduced CEM cells and (▪) SFCMM 3 transduced TK-CEM sub-clones.

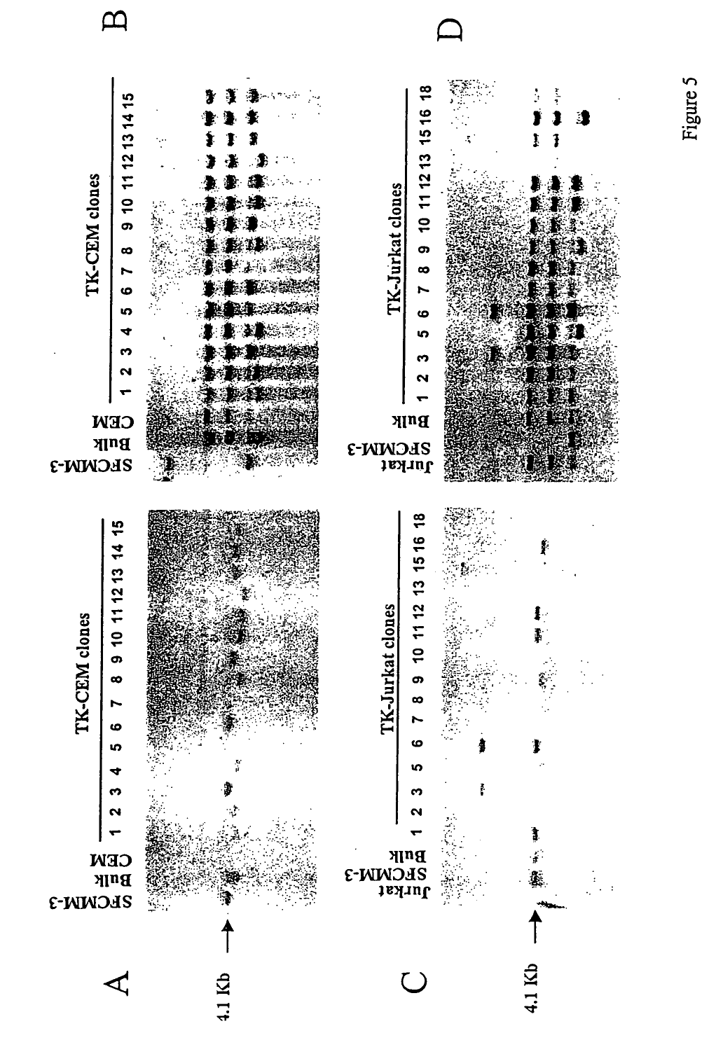

FIG. 5 shows a Southern blot analysis of the TK-CEM (A and B) and TK-Jurkat (C and D) sub-clones. Genomic DNAs were digested with SacI restriction enzyme. The Southern blots were hybridised with TK (A and C) and ΔLNGFR (B and D) specific probes.

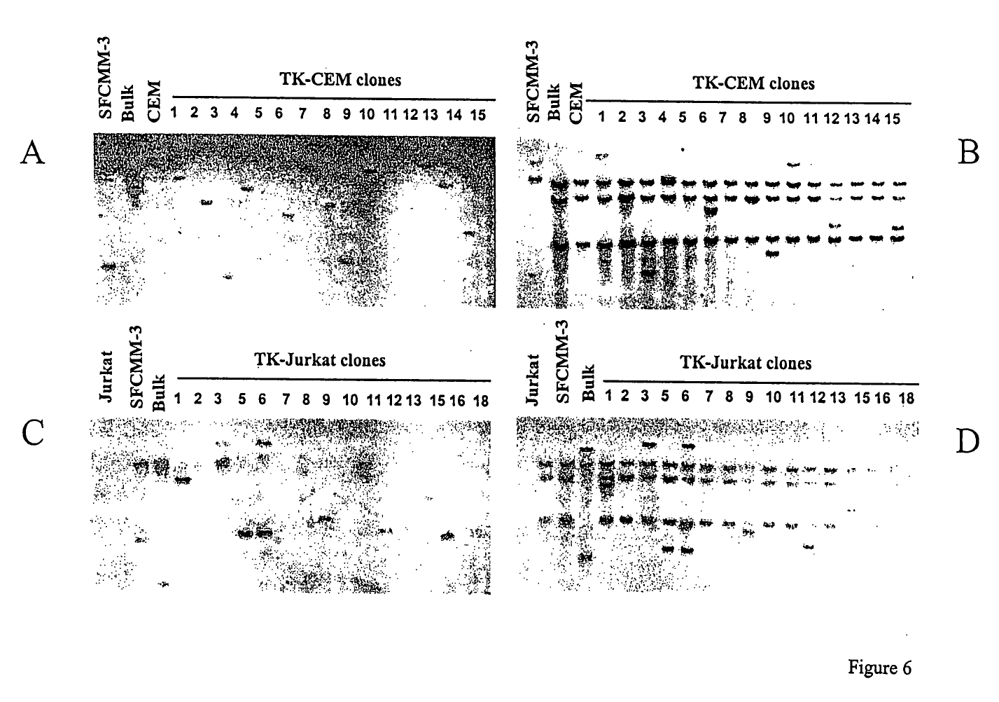

FIG. 6 shows a Southern blot analysis of the TK-CEM (A and B) and TK-Jurkat (B and C) sub-clones. Genomic DNAs were digested with EcoRI restriction enzyme. The southern blots were hybridised with TK (A and C) and ΔLNGFR (B and D) specific probes.

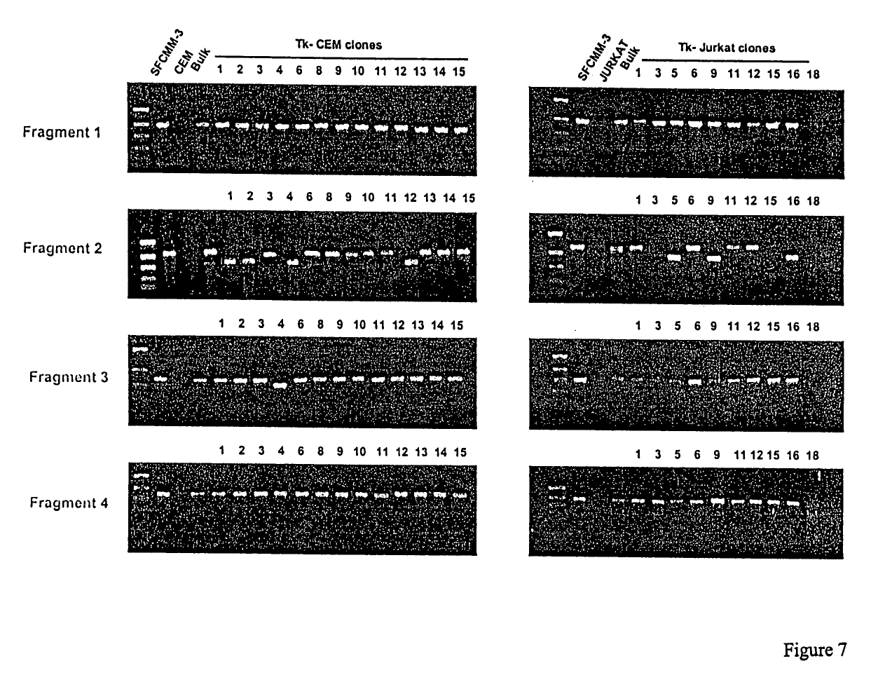

FIG. 7 shows a PCR amplification of SFCMM3 provirus sequence from genomic DNA derived from the TK-CEM and TK-Jurkat sub-clones. Four different regions of the provirus were amplified by PCR using specific primers (see FIG. 1). Fragment 1: LTR1⁺/HTK5⁻ (912 bp); fragment 2: HTK4⁺ and HTK1⁻ (998 bp); fragment 3: HTK1⁺ and NGF2(753 bp) and fragment 4: NGF2⁺ and NGF3⁻ (852 bp).

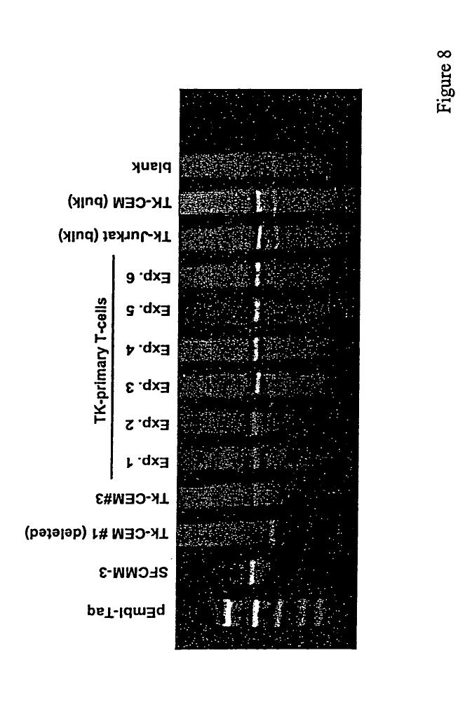

FIG. 8 shows a PCR amplification of HSV-tk gene sequence (fragment 2: HTK4⁺/HTK1⁻; 998 bp) from genomic DNA obtained from transduced and selected primary T-lymphocytes. Positive and negative controls from the TK-CEM and TK-Jurkat sub-clones were used.

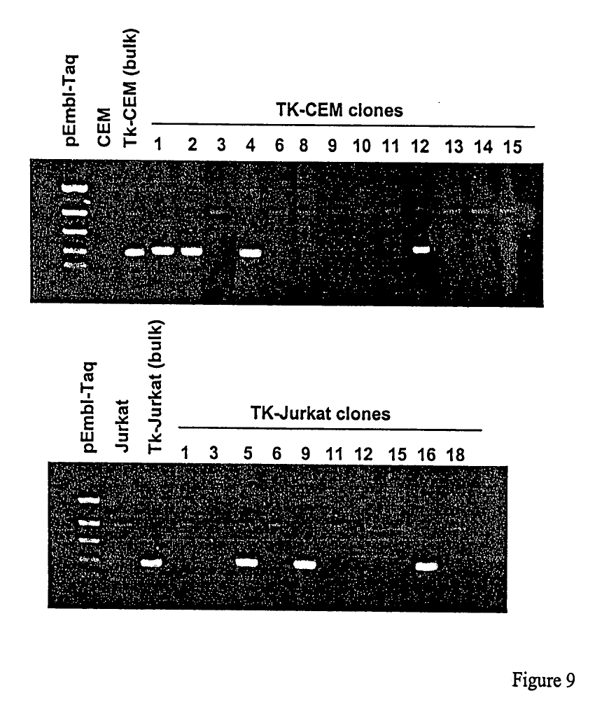

FIG. 9 shows a PCR amplification of the truncated HSV-tk gene from genomic DNA derived from the TK-CEM and TK-Jurkat sub-clones. The PCRs were set up using primers (HTK8⁺/HTK1⁻; 640 bp) that specifically amplified the spliced form of the HSV-tk gene in the GCV-resistant TK-CEM and TK-Jurkat clones.

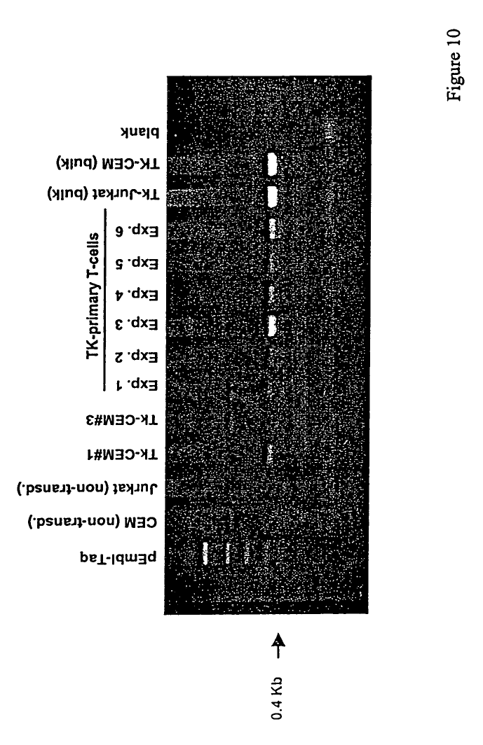

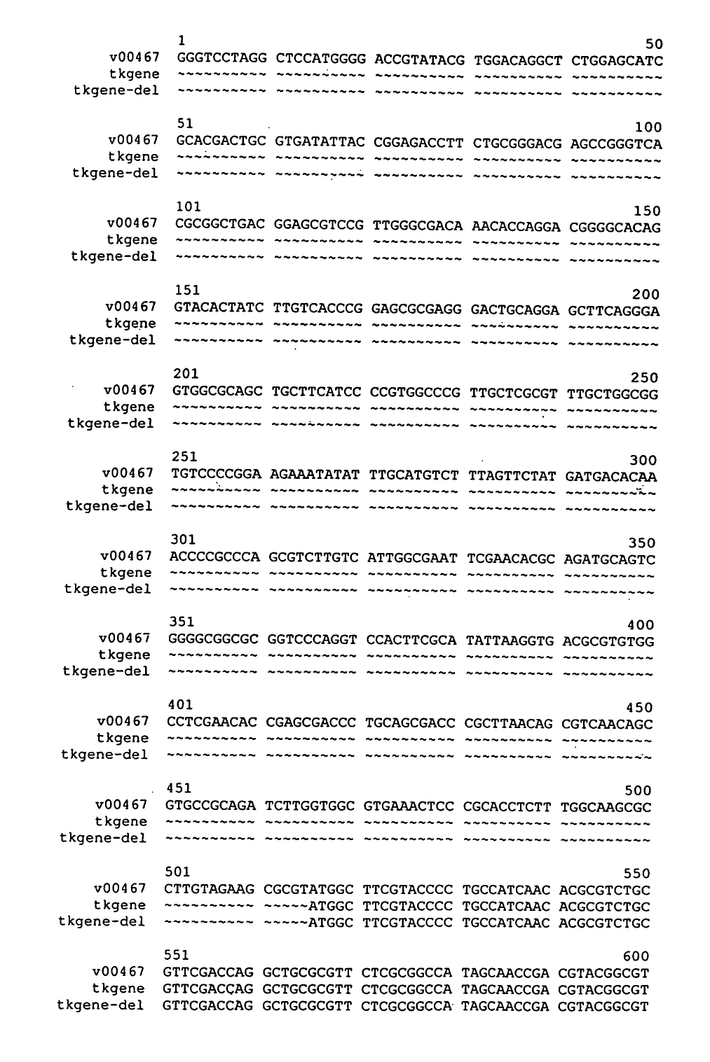

FIG. 10 shows a PCR amplification of the truncated HSV-tk gene from genomic DNA obtained from transduced and selected primary T-lymphocytes. The PCRs were set up using primers (HTK8⁺/HTK1⁻; 640 bp) that specifically amplified the spliced form of the HSV-tk gene in the GCV-resistant TK-CEM and TK-Jurkat clones.

FIG. 11 shows the wild-type sequence of the HSV-tk gene (referred to as V 00467), the full-length HSV-tk gene (tkgene) in the expression vector used in the Example and the deleted gene (tkgene-del) found in the experiments described in the Examples.

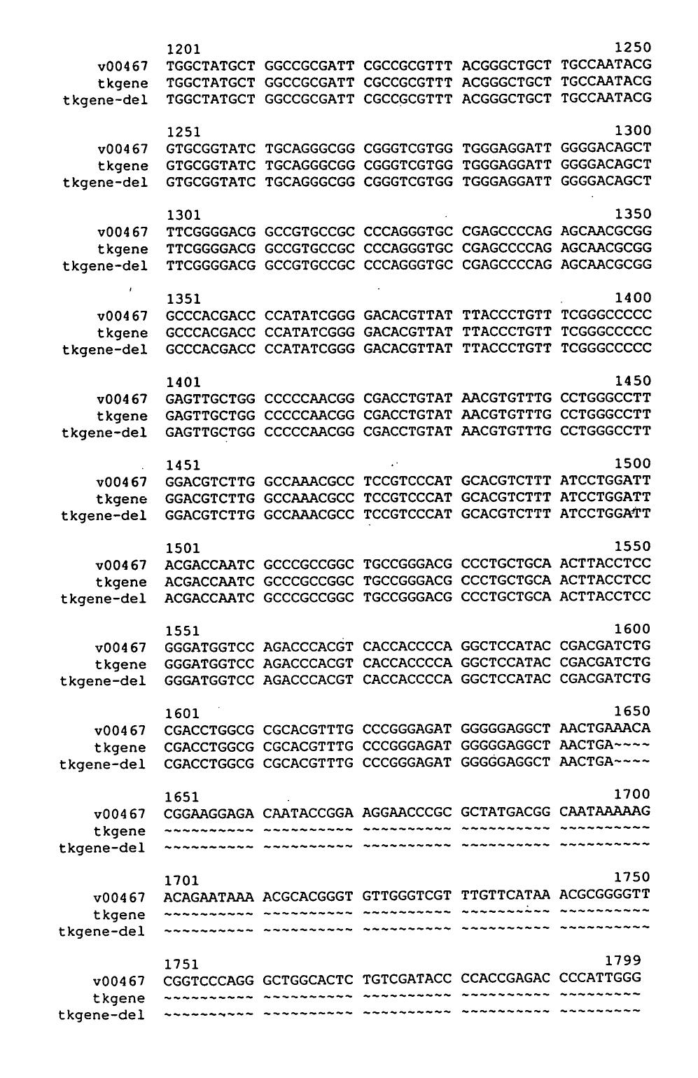

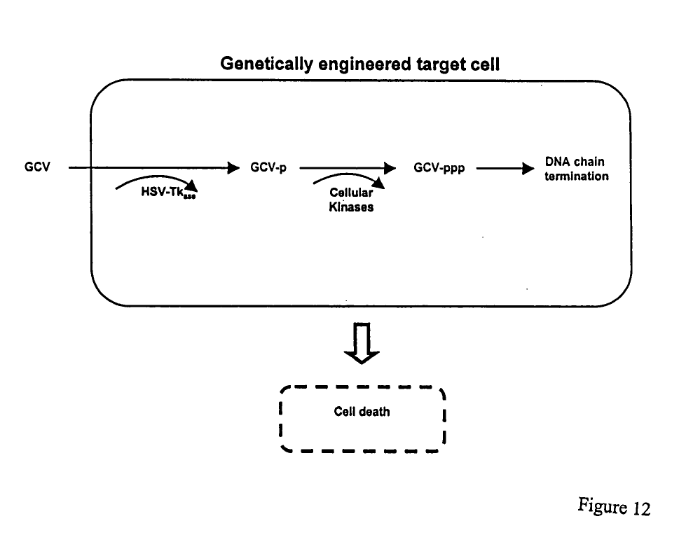

FIG. 12 shows a schematic representation of the HSV-tk/GCV system proposed for killing tumour cells in cancer gene therapy and donor T-lymphocytes for modulation of alloreactivity after bone marrow transplantation.

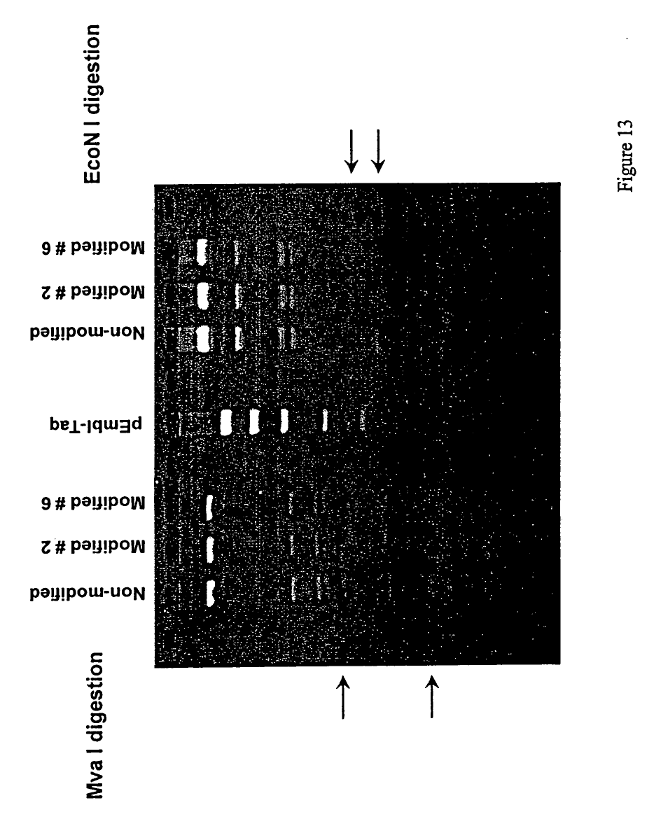

FIG. 13 shows the modification of the mSFCMM-3 vector by site-directed mutagenesis: restriction enzyme analysis of transfectants #2 and #6. EcoN I and Mva I restriction enzymes were used to detect the mutations induced at positions 1994 and 2221 of the vector, respectively. Non-modified vector containing the wild-type HSV-tk gene sequence was used as negative control.

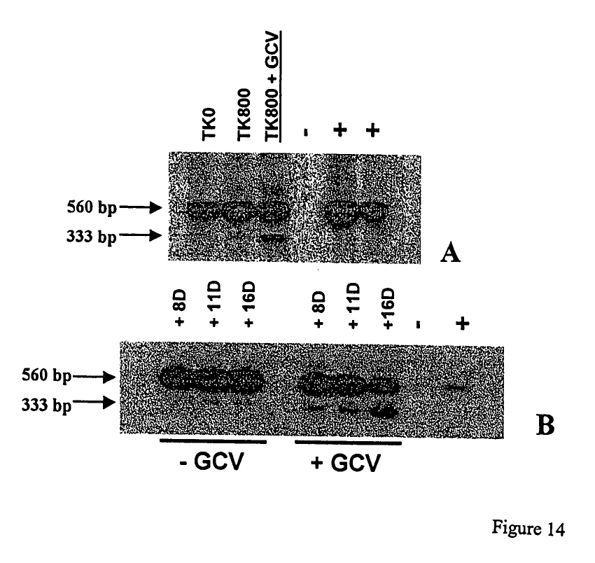

FIG. 14. Southern blot of pTK/RTK2 PCR (35 cycles) products, from transduced primary T-cells in culture with or without GCV. (A) PCR amplification of the HSV-Tk sequence in transduced/unselected T-cells (TK0), transduced/G418 selected T-cells (TK800) cultured in the absence of GCV and with 1 μg/mL GCV for 7 days (TK800+GCV). (B) Transduced primary T-cells after 8, 11, 16 days of culture in presence (1 μg/ml) or absence of GCV. Positive (DNA from G1Tk1SvNa vector producer cells) and negative controls (non transduced primary T-cells) were used.

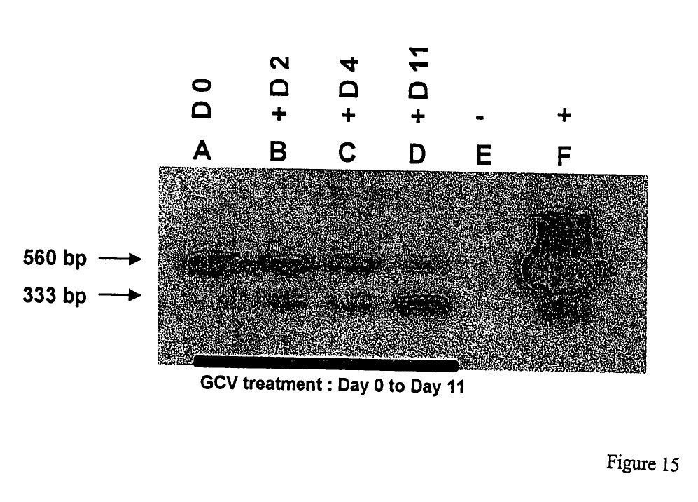

FIG. 15. Southern blot analysis of TK PCR products of peripheral blood mononuclear cells from a representative patient of the clinical trial at time of GvHD (Day 30 post allograft) during GCV treatment. Lanes A-D: DNA samples extracted from PBMCs of the patient at time of GVHD (A), 2 days (B), 4 days (C), 11 days (D) after the beginning of the GCV treatment. Lane E: Negative control (non transduced primary T-cells); Lane F: Positive control (DNA from G1Tk1SvNa vector producer cells).

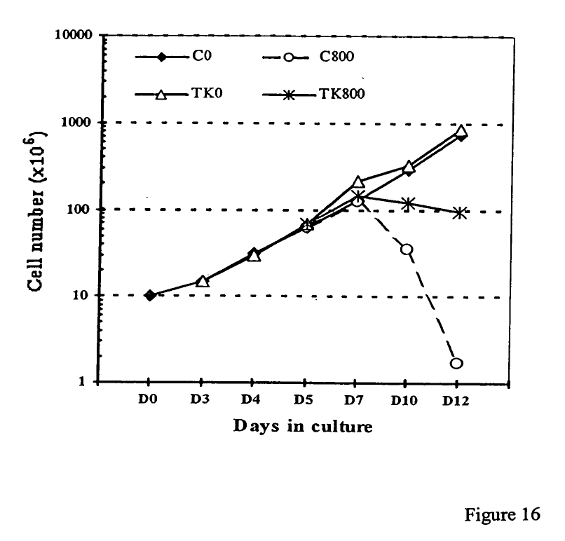

FIG. 16 shows the results of a representative experiment showing relative cell growth of different cell populations transduced with the G1Tk1SvNa vector. C0: non transduced, non selected cells; C800: non transduced cells selected with G418 (800 mg/ml); TK0: transduced, non selected cells; TK800: transduced and G418-selected.

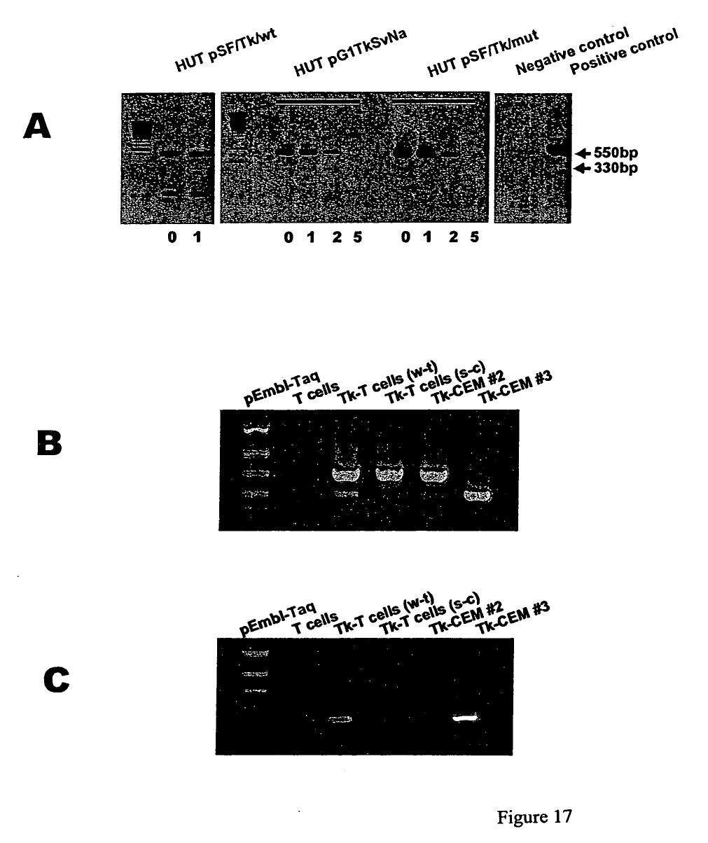

FIG. 17. PCR analysis of T cell lines and primary T cells transduced with corrected or non-corrected HSV-Tk vectors. (A) Southern blot analysis of HSV-Tk PCR products from Hut-78 Cell lines, in the presence of increasing GCV concentrations (0, 1, 2 or 5 μg/ml), transduced with non-corrected vectors G1Tk1SVNa or SF/Tk/wt, or with the corrected vector pSF/Tk/mut. (+) and (−) are the positive and negative controls of the PCR reaction.

MW represents the molecular weight markers with the bright band equalling 600 bp (100 bp DNA ladder, Life Technologies). Amplification by PCR using primers for both forms of the HSV-Tk gene (white arrow=full length and black arrow shows truncated gene. (B) or using primers specific for the truncated form of the HSV-TK gene. (C) on bulk populations of primary T cells transduced with the non corrected SCFMM3 (wt) or with the corrected sc-SCFMM3 (mut) vectors. Tk-CEM #2 and Tk-CEM #3 represent non-truncated and truncated PCR controls. Untransduced Tcells were used as the negative control.

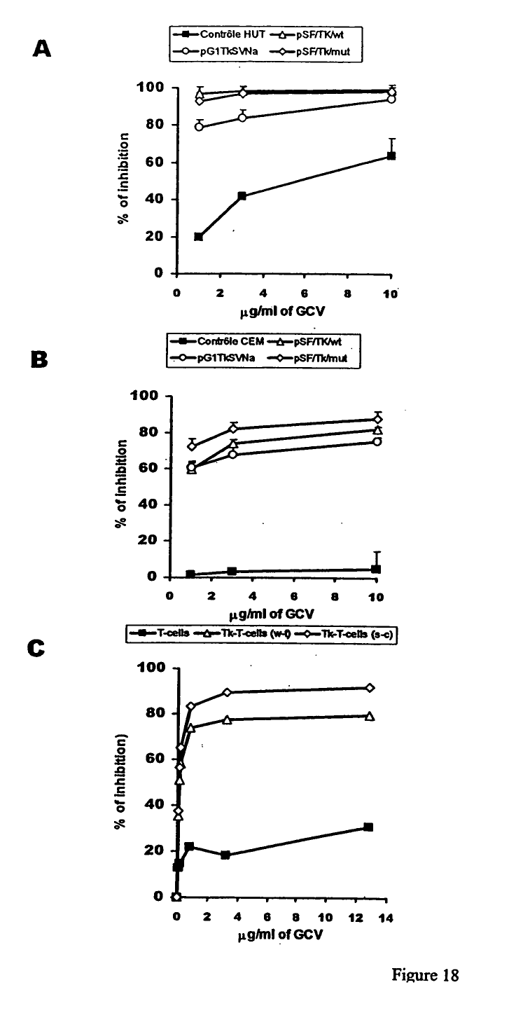

FIG. 18. GCV sensitivity of T cell lines and primary T cells transduced with corrected or non-corrected HSV-Tk vectors. GCV sensitivity of Hut-78 (A) and CEM (B) cell line transduced with corrected SF/Tk/mut (?) or non-corrected vector G1Tk1SVNa (◯) or SF/Tk/wt (Δ) compared to untransduced T-cell lines (▪). The data represents the inhibition of cell viability and are the mean±SD of 8 and 3 different independent experiments, for CEM and Hut-78 cell lines respectively. (C) GCV sensitivity of primary T cells transduced with corrected sc-SFCMM3 (Δ) or non corrected SFCMM3 vectors (?) compared to untransduced T cells (▪).

### EXAMPLE 1

### Molecular Mechanism for Ganciclovir Resistance in Human T-lymphocytes Transduced with a Retroviral Vector Carrying the Herpes simplex Virus Thymidine Kinase Gene

Here we investigate the mechanisms participating in the GCV resistance in transduced CEM and Jurkat cells, two lymphoblastoid human T-cell lines. The retroviral vector used (SFCMM3) contains the HSV-TK gene under the 5′ LTR control and the ΔLNGFR gene which is regulated by the SV40 promoter (Verzeletti et al (1998) Human Gene Ther. 9, 2243-2251). Fifteen sub-clones derived from transduced and selected CEM and Jurkat cells were characterised for the expression of the HSV-tk and ΔLNGFR transgenes. Our results showed that within the sub-clones expressing the ΔLNGFR gene, some GCV resistant sub-clones were identified. The molecular mechanism underlying the GCV resistance involved the deletion of a 227 bp fragment in the HSV-tk gene sequence. Mapping of the truncated HSV-tk gene showed that the deletion was caused by cryptic splicing of vector RNA in producer cells within the HSV-tk sequence. The deleted HSV-tk gene found in some of the subclones is associated with recurrence of HSV-tk gene for GCV. These findings may explain the observations made in a number of previous studies using the HSV-tk/GCV approach in cancer gene therapy and allo-BMT.

Material and Methods

Retroviral Vector and Producer Line

The SFCMM3 vector, provided by Dr. Cl. Bordignon (Milan, Italy), has been described previously (Verzeletti 1998). Briefly, the retroviral vector contains the entire HSV-TK gene sequence under long terminal repeat (LTR) transcriptional control and the ΔLNGFR under the control of an internal promoter SV40. Vector DNA was transfected into E86 ecotropic packaging cell line by calcium phosphate coprecipitation. The supernatants obtained from the transfected E86 cells were used to infect the GP+env Am12 amphotropic cell line. The expression of the ΔLNGFR in the transduced Am12 cells was assessed by FACS analysis using a murine anti-human ΔLNGFR monoclonal antibody (HB 6787, clone 20.4, ATCC, Rockville, Md.) and a FITC labelled goat-anti-mouse IgG₁ monoclonal antibody (Becton-Dickinson, Mountain View, Calif.) as secondary antibody. The retroviral producer clone identified as SFCMM3#16 used in our experiments was maintained in Dulbecco's modified Eagle's medium (GIBCO-BRL, Gaithersburg, Md.) supplemented with 10% heat inactivated fetal calf serum (FCS, Harlan Sera-Lab Ltd., Loughborough, UK), 20 mM L-glutamine, 100 μg/mL streptomicin, 100 U/mL penicillin (GibcoBRL; Life Technologies, Scotland).

Harvesting of Virus Supernatant

Producer cells are maintained 37° C. for their expansion. When the cultures are 90% confluent, the supernatant is replaced with fresh D-10 medium and cells are kept at 32° C. for 16 h. The virus-containing supernatants are harvested and filter through a 0.45 μm mesh to remove detached producer cells and cellular debris. The supernatants are snap-frozen into liquid nitrogen and then stored at −80° C. until use. Viral titers were estimated by the infection of NIH-3T3 cells with serial 10-fold dilutions of virus-containing supernatant and subsequent FACS analysis.

Isolation and Culture of Human Primary T-Cells Lymphocytes and T-Cell Lines

Peripheral blood mononuclear cells (PBMNCs) were obtained in heparinized tubes from healthy donors. Low-density MNCs (<1.007 g/mL) were isolated by centrifugation (1500 g, 30 min, 20° C.) on Lymphoprep (Nycomed, Oslo, Norway). PBMNCs were cultured at a density of 2×10⁶ cells/mL in T-RF10 medium composed by RPMI (GibcoBRL; Life Technologies, Scotland) containing 10% heat inactivated FCS, 5 μM β-mercaptoethanol, 25 μM Hepes both from Sigma (St Louis, Mo.), glutamine, 100 μg/mL streptomycin, 100 U/mL penicillin and 100 U/mL recombinant human interleukin 2 (rhIL-2) from Research & Development System Europe Ltd. (Abingdon, UK) and Prepotech EC Ltd. (London, UK) (T-RF10). CEM and Jurkat, two human lymphoblastoid T-cell lines, were cultured in RPMI supplemented with 10% heat inactivated FCS, 20 mM glutamine, 100 μg/mL streptomycin and 100 U/mL penicillin.

Transduction of Primary T-lymphocytes and Human T-Cell Lines

PBMNCs were stimulated with 1 μg/mL PHA and 100 U/mL rhIL-2 for 48 h. Non-adherent cells were collected by centrifugation and resuspended in T-RF10 at 2×10⁶ cells/mL. CEM and Jurkat cells were fed with RF10 24 h. before the infection. Cell-free virus supernatants obtained from SFCMM3 producer cells and containing 4 μg/mL polybrene (Sigma; St Louis, Mo., USA) were added to the cell cultures and incubated for 16 h. at 37° C. in a CO₂ incubator. The infections were repeated during two consecutive days. Cells were washed with fresh medium 24 h. after the last infection. Transduced cells were further cultured for 2-3 days before the analysis by FACS for the determination of the gene transfer efficiencies.

FACS Analysis for the Determination of ΔLNGFR Expression

Transduced cells were incubated with the non-conjugated murine anti-human ΔLNGFR monoclonal antibody (HB8737, clone 20.4, ATCC) for 40 min. at room temperature. Cells were washed two times with 1% BSA-PBS and stained with goat anti-IgG₁ mouse FITC-couple antibody (Becton-Dickinson, Mountain view, Calif.) for 20 min. at 4° C. For dualcolor analysis cells were stained with PE-couple anti-CD3 monoclonal antibody (MoAb) (Becton-Dickinson, Mountain view, Calif.) for 20 min. at 4° C. and washed twice with 1% BSA-PBS. Finally, the cells were fixed with paraformaldehyde-PBS buffered solution. FACS analysis was performed in a flow cytometer (FACS Scan; Becton-Dickinson). ΔLNGFR expression was measured within the CD3 positive population using the CellQuest© software (Becton-Dickinson). For each reading at least 20,000 events were counted.

Selection of Transduced Cells by Magnetic Sorting

Transduced cells were selected based on the expression of the ΔLNGFR on the cell surface using the MiniMACS system according to the manufacturer's instructions. For the immunoselection, cells were incubated with the murine anti-human ΔLNGFR MoAb for 40 min at room temperature. Cells were washed with MACS buffer (PBS supplement with 0.5% BSA and 2 mM EDTA) and incubated with a goat anti-mouse IgG microbeads (MACS, Miltenyi Biotec, Germany), for 15 min at 4° C. After washing the cells, ΔLNGFR expressing cells were selected over a MiniMACS MS⁺ separation columns (MACS, Miltenyi Biotec, Germany).

Cloning of the Transduced T-Cell Lines

The sub-cloning of the transduced and selected human T-cell lines was performed by plating 400 cells in 1 mL of methyl cellulose (MethoCult H4330; Stem Cell Technologies, Vancouver, Canada) in 35 nun Petri dishes. Semi-solid cultures were incubated for 12 days at 37° C. in a CO₂ incubator. On day 13, colonies were picked and seed in 100 μL of RF10 into a 96-well plate. The growth of the clones was monitored under the microscope. The clones were expanded by adding fresh medium when the supernatants turned yellow and by transferring the clones into vessels of increasing volumes keeping the cell density bellow 1×10⁶ cells/mL.

Ganciclovir Cytotoxic Assay

A total of 2×10⁴ cells/well were seeded into a 96-well plate in 100 μL of medium. Cells were cultured for 4 days with increasing concentrations (from 0.05 to 12.5 μg/mL) of GCV (Cymevene®, Hoffman-La Roche A G, Germany). Afterwards, 1 μCi/well of tritiated thymidine (methyl ³H-thymidine, TRA.120, 1.0 MBq/mL, Amersham International, England) was added 18 h. before harvesting the cell DNA in a cell harvester (Wallac, Gaitherburg, Md.). The incorporation of ³H-thymidine into the DNA was measured in a β-scintillation counter (Wallac 1410, aitherburg, Md.). All GCV concentrations were tested in triplicate esults are expressed as percentage of incorporated ³H-thymidine at each GCV concentration with respect to the incorporation obtained in the absence of GCV.

Southern Blot Analysis

Genomic DNAs were extracted using a QIAamp Blood kit (Qiagen Ltd. Germany) following the recommendations supplied by the manufacturer. After overnight digestion of 10 μg genomic DNAs with the restriction enzymes Sac I or EcoR I (New England Biolabs Ltd.; UK) samples were size-fractionated by electrophoresis through 0.8% agarose gel. DNAs were transferred onto a Hybond N nylon filter (Amersham des Ullis, France) according to the supplier's instructions. The blots were hybridised with α-³²P-dCTP random prime-labelled 1.1 kb Mlu I-Xho I fragment for the HSV-TK gene sequence and 0.9 kb Rsr II fragment for the LNGFR gene. Finally, the blots were exposed to radiographic film (Biomax, Kodak, USA) for at least 16 h. at −80° C.

Polymerase Chain Reaction (PCR) and Sequencing Analysis

The presence of the SFCMM3 provirus sequences was examined in the genomic DNAs extracted from the transduced cells. Four different regions of the provirus were amplified by PCR using specific pairs of primers (FIG. 1): fragment 1: LTR1⁺ (5′GGTCTCCTCTGAGTGATTGACTA3′) and HTK5⁻ (AACGAATTCCGGCGCCTAGAGAA); fragment 2: HTK4⁺ (TTCTCTAGGCGCCGGAATTCGTT) and HTK2⁻ (ATCCAGGATAAAGACGTGCATGG); fragment 3: HTK1⁺ (CCATGCACGTCTTTATCCTGGAT) and NGF2⁻ (TTGCAGCACTCACCGCTGTGTGT) and fragment 4: GF2⁺ (ACACACAGCGGTGAGTGCTGCAA) and NGF3⁻ (ATAGAAGGCGATGCGCTGCGAAT). PCRs were performed in a 20 μL reaction mixture containing 50 ng of genomic DNA, 1×Taq polymerase buffer with 15 mM MgCl₂ (Boehringer Mannhein Ltd., Lewes, UK), 250 μM each dATP, dCTP, dGTP, dTTP, 0.25 μM each sense and antisense primer, and 0.025 U/μL Taq polymerase (Boehringer Mannbein Ltd.). Thermocycling conditions to amplify fragments 1 and 2 were 35 cycles of denaturation at 96° C. for 30 sec., annealing at 60° C. for 30 sec and extension at 72° C. for 1 min followed by a final 10 min extension at 72° C. Thermocycling conditions used to amplify fragments 3 and 4 were 31 cycles of denaturation at 96° C. for 30 sec, annealing at 64° C. for 50 sec and extension at 72° C. for 1 min, followed by a final 10 min extension at 72° C. The PCR products (10 μL) were electrophoresed on a 1% agarose gels containing ethidium bromide.

PCR amplification of an 880 bp genomic fragment of the human ABL gene was performed as described elsewhere (Melo et al (1994) Leukemia 8, 208-211) on negative clones to confirm the presence of amplifiable genomic DNA.

Cloning of PCR products was achieved using the pCR2.1 TA cloning vector from Invitrogen (Groningen, The Netherlands). Cloning was performed in duplicate from independent PCR reactions. Automated fluorescent DNA sequence analysis using M13 primers was carried out by Advanced Biotechnology Centre (London, UK).

Results

Transduction and Selection of Human T-Cell Lines

CEM and Jurkat cells were transduced following a cell-free virus supernatant infection protocol in the presence of polybrene. The efficiencies of the gene transfer experiments were determined by FACS analysis based on the LNGFR expression on the cell surface. The transduction efficiencies obtained were similar for both cell lines: 20±5 (n=3) for CEM cells and 17±6 (n=3) for Jurkat cells (FIGS. 2A and C). The selection of the transduced cells was performed using an immunomagnetic procedure (MACS System). After selection, 85% for CEM cells and 83% of Jurkat cells expressed the LNGFR (FIGS. 2B and D). The enrichment in LNGFR expressing cells can be further improved up to 95-98% by performing a second round of selection.

ΔLNGFR Expression in TK-CEM and TK-Jurkat Clones

Transduced and selected CEM and Jurkat cells were cloned by plating the cells in semi-solid media. On day 10, the colonies were picked and transferred to a 96-well plate for further expansion of the sub-clones in liquid cultures. Six weeks later, when enough cells were available, the expression of the LNGFR gene was determined by FACS. FIG. 3 shows the histograms obtained for each of the sub-clones. The majority of the clones derived from transduced CEM cells were positive for ΔLNGFR (12 out of 15). TK-CEM clones #5 and #7 showed no expression for the ΔLNGFR as demonstrated by the overlapping profiles with respect to the isotopic controls (FIG. 3A). The FACS analysis performed on the TK-Jurkat derived clones revealed that seven of the fifteen sub-clones analysed were positive and eight were negative (FIG. 3B). The TK-Jurkat clone 12 showed a double shoulder indicating that two different sub-clones might be participating in this cell line.

Different levels of expression of the ΔLNGFR were observed within the clones expressing the ΔLNGFR reporter gene. The expression of the ΔLNGFR in the TK-Jurkat #3 and #11 was two orders of magnitude higher than TK-Jurkat #4 and #6. ΔLNGFR expression ranged in only one order of magnitude within the TK-CEM clones (FIG. 3A). Overall, TK-Jurkat clones showed a wider range for the expression of ΔLNGFR than the sub-clones derived from the transduced CEM cells.

HSV-TK Gene Expression in TK-CEM

The expression of the HSV-TK transgene m the TK-CEM sub-clones was determined by the inhibition in cell proliferation when the cells were cultured at increasing concentrations of GCV. FIG. 4 shows the result obtained in the GCV-induced cytotoxic assay performed in the TK-CEM clones. The IC₅₀ inhibition in cell proliferation assessed by the incorporation of ³H-thymidine was reached at 0.1 μg/mL GCV concentrations for clones #3, #8, #9, #11, #12, #13 and #15. In contrast, the cell proliferation in clones #1, #2, #4, #5, #7 and #14 was parallel to that obtained in the non-transduced cells (negative control). TK-CEM clones #6 and #10 required higher concentration of GCV (0.25 μg/mL) to achieve the 50% inhibition in cell proliferation. In these two instances, complete inhibition in cell proliferation was not completely achieved when the concentration of GCV was increased up to 12.5 μg/mL.

Integrity of the Inserted SFCMM3 Provirus

Genomic DNA extracted from the CEM and Jurkat clones was examined for the integrity of the inserted SFCMM3 provirus by Southern blot analysis. Sac I digested genomic DNAs were subjected to gel-electrophoresis and then transferred to a membrane. The blots were hybridised successively with specific probes for the HSV-TK and LNGFR genes (FIG. 1). The Sac I restriction enzyme cuts only at the 5′ and 3′ LTRs. Southern blot analysis should result in a single band of 4.1 kb size using either of the two probes. FIG. 5A shows that thirteen of the fifteen TK-CEM derived clones had the integrated provirus when the blots were hybridised with the TK-probe. However, only in six cases (clones #3, #6, #9, #13, #14 and #15) the size of the band is the same as in producer cells. An approximately 200-bp smaller band was observed in TK-CEM clones #1, #2, #4, #8, #10, #11 and #12. In the uncloned bulk TK-CEM cells a smear was obtained from the 4.1 kb position. When a specific probe for ΔLNGFR was used (FIG. 5B), sequences corresponding to endogenous NGFR gene appeared in all instances including the non-transduced parenteral CEM cells indicating that all the genomic DNAs were properly digested and homogeneously distributed along the gel. An additional band of the same size as those observed using the TK-probe is observed in the clones carrying the provirus. The absence of the 10 SFCMM3 vector sequences was confirmed using either of the TK and ΔLNGFR probes in the TK-CEM clones #5 and #7. The size of the additional band in the TK-CEM clones #3, #6, #9, #13, #14 and #15 is similar to that obtained in the producer cells.

The Southern blot analysis performed on the genomic DNA extracted from TK-Jurkat clones is shown in FIG. 5C. TK-Jurkat clones #1, #11 and #12 have a band of the expected size with the TK-probe. TK-Jurkat clone #6 has one band of the expected size and a larger band at 15 kb similar to the one observed in TK-Jurkat clone #3. TK-Jurkat #15 showed also one single band of 18 kb size. These observations were confirmed using the LNGFR-probe (FIG. 5D). As in the TK-CEM clones, the bands corresponding to the endogenous NGFR also appeared. Additional bands corresponding to the provirus were observed at the same position as those found using the TK-probe. TK-Jurkat clones #2, #7, #8, #10, #13 and #18 were negatives for provirus sequences.

Number of Insertions and Integration Site of the SFCMM3 Provirus Sequence

To determine the number of insertions and the integration site for the vector in each clone, genomic DNAs extracted from the TK-CEM and TK-Jurkat clones were digested with the restriction enzyme EcoR I that has only one restriction site within the provirus sequence (FIG. 1). Southern blot analysis using the TK-probe showed that in the clones derived from the transduced CEM cells, different position of the TK-containing fragments was observed. This suggests-single and independent integration events into random sites within the cell genome (FIGS. 6A and B). Similar results are observed for the EcoR I digested genomic DNAs derived from the TK-Jurkat clones (FIGS. 6C and D). TK-Jurkat #6 has two bands at different positions indicating multiple integration sites of the provirus into the cell genome.

PCR Amplification of Provirus Sequences

Primers were designed along the SFCMM3 vector to amplify by PCR the whole sequence of the provirus (FIG. 1). PCR analysis of genomic DNAs extracted from the TK-CEM and TK-Jurkat clones amplified bands of expected sizes when specific primers were used to amplify the fragment 1 (LTR1+/HTK5−; 0.93 kb), fragment 3 (HTK1+/HTK2−; 0.77 kb) and fragment 4 (HTK1+/NGF2−; 0.87 kb). For fragment 3, a smaller band than the one amplified in the producer cells (positive control) was obtained in only one case (TK-CEM #4). The bands corresponding to the HSV-TK gene sequence (fragment 2) were the expected ones for TK-CEM clones (#3, #6, #8, #9, #10, #11, #13, #14 and #15) and TK-Jurkat clones (#1, #6, #11 and #12). Smaller bands of the same size were amplified for the TK-CEM clones (#1, #2, #4 and #12) and TK-Jurkat clones (#5, #9 and #16). In the uncloned transduced CEM and Jurkat cells (bulk populations) two bands were amplified of the same sizes as the ones amplified in each single clone. Two of the fifteen TK-CEM clones (#5 and #7) and nine out of fifteen TK-Jurkat clones (#2, #4, #5, #7, #8, #10, #13, #14 and #18) did not amplify any sequence of the provirus by PCR (results not shown). These results agree to those previously observed by Southern blot analysis and expression analysis of the HSV-TK and LNGFR genes.

DNA Sequence Analysis of the Integrated Provirus

To further analyse the provirus sequences, DNA fragments containing the HSV-TK gene (fragment 2) were amplified from genomic DNA. The resulting fragments were cloned into TOPO-A vector to be subsequently sequenced. The results of the sequence analysis performed on some of the clones showed that the small bands amplified by PCR (FIG. 7) resulted from the deletion of 228 bp within the HSV-tk gene sequence. In the cases analysed, the junction region arose from the joining of a cryptic donor site and cryptic splice donor site (CAGG/GTGA, at position 1994 of the retroviral vector) and a cryptic splice acceptor site (CCAG/GCCG, position 2221 of the SFCMM3 vector). This observation was confirmed in both transduced T-cell lines.

PCR on Transduced Primary T-lymphocytes

The retroviral vector used was initially developed for a multi-center clinical trial involving the transduction of primary T-lymphocytes. Concerning the efficacy of the retroviral vector for clinical use we analysed by PCR the HSV-TK gene region of the provirus from genomic DNA extracted from transduced and selected human primary T-lymphocytes. FIG. 8 shows the results of the PCR set up in six different experiments. Positive and negative controls were also established in parallel using some of the TK-CEM and TK-Jurkat clones. One single band corresponding to the full-length HSV-TK gene was amplified by PCR in the transduced primary T-cells. TK-CEM and TK-Jurkat bulk populations showed a smaller band of the expected size corresponding to the truncated form of the HSV-TK gene. These results might indicate that the deletion observed in the HSV-TK gene in some of the clones might occur only in transformed cells. Another explanation for this observation could be that the frequency of the deletion of the HSV-tk gene in transduced primary cells is below the detection levels of the PCR.

Specific Amplification of the Truncated HSV-TK Gene in TK-CEM and TK-Jurkat Clones

A primer was designed at the deletion junction of the HSV-TK gene to specifically amplify the truncated HSV-TK gene found in some of the TK-CEM and TK-Jurkat sub-clones. The size of the amplified band should be 640 bp. FIG. 9 shows the results obtained in the PCR set up using genomic DNAs extracted from the sub-clones. The TK-CEM clones #1, #2, #4 and #12 as well as TK-Jurkat clones #5, #9 and #16 amplified a band of the expected size (640 bp). These are the same clones that amplified a short band for the HSV-TK gene using primers to amplify the full-length HSV-TK gene (FIG. 7). A fainter band of 900 bp was also amplified in all cases including the non-transduced parenteral CEM and Jurkat cells. Modification of the PCR conditions such as the use of increasing annealing temperatures did not improve the specificity of the PCR. We then sequenced the interfering band to eliminate the possibility of having any contamination that could affect the outcome of the PCR. The result of the analysis revealed that the sequence of the interfering band does not correspond to any sequence of the SFCMM3 retroviral vector or to any other sequence represented in the GenBank or EMBL databases (as of January 1999). It probably represents some cellular sequences still uncharacterised.

Amplification of the Truncated HSV-TK Gene in Transduced Primary T-lymphocytes

A PCR using the primer that specifically amplified the truncated HSV-TK gene in the GCV-resistant clones was set up using genomic DNA extracted from transduced and selected primary T-lymphocytes. Positive and negative controls were also set up in parallel. In the six different experiments assessed a band of the expected size for the truncated HSV-tk gene was amplified (FIG. 10). These results indicate that the truncated HSV-TK gene is also present in the transduced primary T-lymphocyte populations. The frequency of this event in primary cells is lower than in the human T-cell lines since it can be observed only when the specific primer to amplify the deletion junction of the truncated HSV-TK gene is used.

Discussion

The Herpes simplex virus thymidine kinase type 1 (HSV-tk) encodes an enzyme able to convert the nontoxic prodrug such GCV and acyclovir into cytotoxic metabolites. Gene transfer strategies using this and other suicide gene/prodrugs systems have been proposed as a novel therapeutic modality for treatment of cancer^{1; 3; 11}. More recently, the use of suicide gene therapy has been extended for allogeneic bone marrow transplantation (allo-BMT). Donor T-lymphocytes transduced with the HSV-tk gene can be selectively removed from circulation by administration of GCV. This would allow the modulation of the GvHD while preserving a significant GvL and immune reconstitution^{8; 9}.

The retroviral vector used in our study contains the ΔLNGFR gene that works as selectable marker for the cells expressing the provirus. The ΔLNGFR cDNA was modified in such a way that is biologically unable to bind to nerve growth factor and to trigger any transduction signal through the cytoplasmic domain¹². The use of ΔLNGFR as reporter gene has been proposed to monitor the transduction efficiency and to facilitate the selection of the transduced T-lymphocytes in a shorter time frame than the commonly used systems based on the expression of other genes such as the neomycin resistant gene^{13; 14}. Additionally, genetically modified cells can be easily tracked and possibly reselected after infusion into patients.

The efficacy of the HSV-tk/GCV system has been demonstrated in a number of in vitro and in vivo models. It has also been shown that the use of this system for the treatment of cancer offers additional advantages derived from the so-called bystander-effect. In this situation HSV-tk transduced cells together with the non-transduced neighbouring cells are killed following administration of GCV. The mechanism underlying this event is thought to be mediated by transfer of phosphorylated GCV from transduced tumor cells to non-transduced cells via gap junctions¹⁵. The bystander-effect has been shown to be essential for the complete regression of the tumor in which only a fraction of the cells in the tumor mass are transduced¹⁶. Tiberghien et al (1994)¹⁷ demonstrated that in transduced primary T-cells GCV-induced growth inhibition is not mediated through a bystander-effect. They examined the effect of GCV in a mixture of transduced and non-transduced T-cells (50/50) as well as in HUT-78 cells (CD4⁺ mature T-cell lymphoma cell line). In both instances they found no evidence for a bystander-effect mediated killing. The absence of bystander-effect in HSV-tk T-lymphocytes has important implications for the successful application of suicide gene strategies in the context of allo-BMT since it will allow the depletion from circulation specifically of those cells responsible for the GvHD.

The use of the HSV-tk gene/GCV system was first proposed by Moolten and Wells (1986)⁴. Later on, they also observed recurrence of HSV-tk transduced sarcoma and lymphoma tumor cells to GCV treatment in in vivo studies^{2; 18}. Similar observations have been confirmed in tumor cells derived from different tissues and animal models. Barba et al (1993)¹⁹ described that genetically modified rat glioma cells expressing the HSV-tk gene were killed in culture following 14 days of GCV treatment. Eventually, some modified tumor cells became resistant to GCV. However, due to the bystander-effect, the inactivation of the HSV-tk gene did not interfere in the outcome of the in vivo assays in which the GCV treatment resulted in additional killing of non-transduced tumor cells in the brain. Similar observations have been described in human adenocarcinoma cell lines. In vitro studies revealed that cells derived from epithelium showed adequate expression of the HSV-tk transgene. In contrast, tumor regression following GCV treatment was not observed in nude mice bearing HSV-tk transduced adenocarcinoma cells. In this in vivo model, the lack of bystander-effect did not contribute to abrogate the faulty expression of the HSV-tk gene in a small subset of transduced tumor cells.

The mechanisms underlying the lack of expression of the HSV-tk gene in transduced tumor cells are poorly understood. Several explanations as changes in cell susceptibility to GCV, transient exit of tumor from cell-cycle, inactivation or loss of HSV-tk gene or structural changes in the HSV-tk mRNA and transient methylation events have been proposed in the literature. Di Ianni et al (1997)²⁰, reported the lack of HSV-tk transgene expression in clones derived from U937 cells (a human haemopoietic malignant cell line) genetically engineered to express the HSV-tk gene and the bacterial β-galactosidase gene (LacZ gene) in a bicistronic vector. The subclones showing resistance for GCV also failed in the histochemical staining with 5-bromo-4-chloro-3-indolyl-β-galactopyranoside (X-Gal). They cultured the resistant clones with 5-azacytidine (a dimethylating agent). This treatment could not restore the expression of either of the two transgenes. The authors also opened the possibility for rearrangements or mutations at the LTR of the bicistronic vector.

The GCV resistance in human primary T-lymphocytes transduced with a retroviral vector carrying the HSV-tk gene has been documented by different groups. Tiberghien et al (1994, 1997)^{9; 17} described between 80% to 90% growth inhibition following GCV-treatment of IL-2 responding transduced and neomycin selected T-cells. These results may suggest that at least 10% of the transduced populations are resistant to killing by GCV. In the first clinical trial using transduced donor T-cells Bonini et al. (1996)¹⁰ observed that one patient developed chronic GvHD. After administration of GCV the proportion of transduced donor cells declined from 11.9% to 2.8%. Complete depletion of transduced donor derived T-cells from circulation could not be achieved following an intensive treatment with GCV. This would mean that about 23% of donor T-lymphocytes lack adequate expression of the HSV-tk gene. Verzeletti et al (1998) further characterised the GCV-resistance observed in the patient that developed chronic GvHD. The authors claimed a cell-cycle dependence of the HSV-tk/GCV system rather than a molecular event occurring in the HSV-tk gene sequence.

Using the retroviral vector we found that some of the sub-clones derived from transduced and selected TK-CEM and TK-Jurkat populations did not show an adequate expression of the HSV-tk gene. The Southern blots revealed a shortened integrated provirus in the cell genome (FIG. 5). The PCR analysis performed in the clones indicated that the molecular events participating in the GCV-resistance took place in the HSV-tk gene sequence of the provirus (fragment 2, FIG. 7). The sequencing analysis has shown that the short form of the HSV-tk gene resulted from a 228 bp deletion in the DNA sequence of the HSV-tk gene. The mapping of the junction region corresponds to the joining of cryptic splice donor site and cryptic splice acceptor site at positions 1993 and 221 of the vector, respectively. These findings may suggest that in the producer cells part of the mRNA from the provirus might be recognised by the splicing machinery of the Am12 cells resulting in spliced vector-derived RNA. This may cause the production of virus particles containing the full-length HSV-tk gene together with a small proportion carrying the aberrant HSV-tk gene. This mechanism explains the passage of the truncated provirus HSV-tk gene to the target cells. Northern blot analysis performed on the TK-CEM clones has revealed the formation of aberrant HSV-tk transcripts in those clones carrying the truncated gene (results not shown). It is likely that a non-functional protein is encoded from the aberrant HSV-tk gene sequence.

The frequency of transduced cells containing the deleted HSV-tk gene seems lower in transduced primary T-lymphocytes than in the transformed T-cell lines. To detect the truncated HSV-tk gene in the transduced primary T-lymphocytes a primer was devised to allow the deletion junction of the spliced HSV-tk gene to be specifically amplified by PCR. This observation may indicate that the virus particles derived from the deleted HSV-tk gene showed lower infective capacity for primary T-lymphocytes than for transformed T-cells. It is also possible that in the transduced T-cell lines those clones containing the aberrant form of the HSV-tk gene have an advantage in proliferation in vitro with respect to the ones having the full-length HSV-tk gene since the functional HSV-tk enzyme is not interfering in any pathway of the cell metabolism.

Yang and colleagues (1998)²¹ found resistant colonies in HSV-tk transduced gastrointestinal tumor cell. The characterization of the resistant colonies demonstrated that the HSV-tk gene was either partially (a 220 bp deletion) or completely deleted from the resistant HSV-tk transduced cells. Interestingly, to rule out the possibility of an unknown cellular mechanism participating in the GCV resistance, they infected the retrovirally transduced-GCV resistant clones with an adenoviral vector containing the HSV-tk gene. The GCV sensitivity was restored, suggesting the capability of these cells to express a functional HSV-tk protein. In contrast to our findings, their results showed that the GCV cytotoxic effect varied among the different gastrointestinal tumor cell lines tested. As has been previously discussed, GCV-resistant transduced cells were not identified in those cell lines having a good bystander-effect.

The in vitro and in vivo data studies as well as the available clinical data suggest that use of the HSV-tk gene/GCV system for cancer gene therapy and allo-BMT represents a realistic improvement for the clinic. However, there are important limitations that need to be solved in the near future. These results strongly support the idea that new suicide genes or optimised versions of the ones currently in use such as the HSV-tk gene should be developed to achieve optimal killing efficiency (100%) of the genetically engineered cells. As shown in Example 2, we have modified the HSV-tk DNA sequence in such a way that it should be ignored by the splicing machinery of the host cells. This strategy allows for the development of an improved HSV-tk gene-containing vector for clinical use.

### EXAMPLE 2

### Modification of the Wild-Type HSV-tk Gene Sequence to Improve the Efficacy of the HSV-tk/GCV System for Suicide Gene Therapy

Suicide genes code for enzymes that render cells sensitive to otherwise non toxic compounds. The thymidine kinase encoded by the Herpes simplex virus type 1 (HSV-tk) converts ganciclovir (GCV) into a metabolite that inhibits DNA elongation. This event, which does not occur in normal cells, leads to cell death. The artificial transfer of the HSV-tk gene into T lymphocytes can therefore provide a system to kill dividing T cells when required. This approach has been exploited and proven effective for treatment of cancer and control of DLI-induced GVHD.

The efficacy of the HSV-tk/GCV system has been demonstrated in a number of in vitro and in vivo models. However, we have found that there is a subset of genetically engineered cells resistant to GCV killing within the transduced population. The mechanism underlying the lack of expression of the HSV-tk gene in transduced cells is poorly understood. We have isolated GCV resistant sub-clones derived from transduced and selected cells. The analysis performed has revealed that the molecular mechanisms participating in an inadequate expression of the transgene involves, at least in some circumstances, a 227-bp deletion in the HSV-tk gene sequence. The mapping of the truncated HSV-tk gene showed that the junction region corresponds to the joining of cryptic splice donor site and cryptic splice acceptor site at positions 844 and 1071 for the 5′ and 3′ ends, respectively (FIG. 11).

Our findings indicate that in the retrovirus producer cells part of the mRNA derived from the provirus is spliced in the vector-derived mRNA. This is believed to cause the production of virus particles containing the full-length HSV-tk gene together with a small proportion carrying the aberrant form of the HSV-tk gene. This mechanism explains the passage of the truncated provirus HSV-tk gene to the target cells.

Our results strongly support the idea that modification of the wild-type HSV-tk gene sequence at the cryptic splice donor and acceptor sites result in an optimised suicide gene. In this case the splicing machinery of the host cells should ignore the engineered-derived mRNA transcripts. The modified HSV-tk DNA sequence was derived from the wild-type sequence of the gene by ablation of cryptic mRNA splicing sites. Two mutations at positions 842 (from GAC CAG GGT to GAC CAA GGT) and 1070 (from CCC CAG GCC to CCC CAA GCC) have been introduced simultaneously by means of enzymatic extension of mutagenic oligonucleotides. In both instances the wild-type amino acid sequence of the HSV-tk enzyme is preserved.

The HSV-tk gene modified by site-directed mutagenesis has been sequenced to confirm the ablation of cryptic splicing sites at desired positions. The expression of the engineered protein in transduced cells is similar to that obtained in those cells transduced with the wild-type HSV-tk gene as shown by inhibition in cell proliferation assays. The PCR developed to specifically amplify the truncated HSV-tk gene in transduced cells showed that the deleted HSV-tk gene was not identified in the cells transduced with the modified HSV-tk gene. We have screened sub-clones derived from cells transduced using the modified gene. The 227-bp deletion in the HSV-tk gene sequence of the vector has not been identified in any of the clones tested.

The mutations at positions 842 and 1070 described above were introduced by the method of Deng and Nickoloff (1992; Anal. Biochem. 200: 81). A commercial kit (Transformer™ Site-Directed Mutagenesis Kit; Clontech, Palo Alto, Calif., USA) was used for the modification of the HSV-tk gene in the retroviral vector. In both instances the wild-type amino acid sequence of the HSV-tk enzyme is preserved. This method allows the specific introduction of base changes into any double-stranded plasmid by means of simultaneous annealing of two or more oligonucleotide primers to one strand of a denatured double-stranded plasmid DNA. In our case two different primers (MUT1+: CCGCCTCGACCAAGGTGAGATATC and MUT2+: CAGCATGACCCCCCAAGCCGTGCTGGCGTTC) were used to introduce the desired mutations (referred to as mutageneic primers). A third primer (selection primer; MUT3+: AGTGCACCATGGGCGGTGTGAAAT) mutates a unique restriction enzyme site (Nde I) in the plasmid for the purpose of selection enzymatic digestion. After standard DNA elongation and ligation, a primary selection was performed by Nde I digestion to partially enrich for the mutated DNA strand. MutS E. coli cells (strain defective in mismatch repair) were transformed using the digested mixture. Plasmid DNA was extracted from the mixed bacterial population and subjected to a second selective Nde I digestion. By this strategy, the parental (non-mutated) DNA is linearised, rendering it much less efficient for transformation of bacterial cells. A final transformation of the thoroughly digested DNA into DH5α bacterial cells was performed. DNA was isolated from individual transformants. The presence of the desired mutations was confirmed first by restriction enzyme analysis in the plasmid DNA extracted from the colonies. EcoN I was used to detect the mutation induced at position 1993 of the retroviral vector. Transformants #2 and # 6 were further characterised for the second mutation at position 2221 of the retroviral vector by Mva I digestion (FIG. 12). Sequencing analysis was also carried out in plasmid DNA isolated from these colonies to verify that other modifications were not introduced in the DNA sequence during the mutagenesis experiment.

### EXAMPLE 3

### Elimination of the Truncated Message From the Herpes Simplex Virus Thymidine Kinase Suicide Gene

The Herpes simplex virus thymidine kinase (HSV-Tk) gene introduced into target cells renders them susceptible to killing by ganciclovir (GCV). We are studying the use of HSV-Tk-transduced T lymphocytes in the context of hematopoietic stem cell transplantation. We have previously shown in vitro and in vivo, the occurrence of transduced cells resistant to GCV due to a deletion within the HSV-Tk gene. This deletion, a consequence of the presence of cryptic splice donor and acceptor sites, originates in the retroviral producer cell. Here we adopt two different methods, which introduce third-base degenerate changes at the cryptic splice sites and so prevent splicing. Consequently, the HSV-Tk protein is unaltered and the sensitivity of the target cells to GCV is preserved. The use of this mutated HSV-Tk gene should reduce the likelihood of the development of resistant genetically modified cells during clinical trials.

We and others are evaluating the use of donor T cells expressing the Herpes simplex virus thymidine kinase (HSV-Tk) gene to modulate alloreactivity after allogeneic hematopoietic stem cell transplantation using transduced T cells.^{1,2,3} The infusion of donor T cells containing the HSV-Tk gene together with a T-cell depleted graft may allow the beneficial maintenance of the graft-versus-leukaemia effect but these T cells can be subsequently eliminated, by GCV administration, at the onset of graft-versus-host disease.⁴ Other groups have similarly used transduced donor T cells to treat Epstein-Barr virus-related lympho-proliferative disease or leukaemia relapse after transplantation.^{2,5,6}

We have shown in vitro and in vivo, the presence of GCV-resistant HSV-Tk transduced human T lymphocytes containing a truncated form of the HSV-Tk gene.⁷ The origin of this truncation was traced to splicing of the gene within the retroviral packaging cells, which was subsequently transmitted to target cells. Molecular analysis of GCV-resistant cells demonstrated the deletion of a 227-bp fragment due to the presence of cryptic splice donor and acceptor sites⁷. Here, we demonstrate that the HSV-Tk gene can be mutated to prevent splicing in the packaging cell line while preserving GCV sensitivity in transduced T cells.

Materials and Methods

Correction of Both Donor and Acceptor Splice Sites by Directed Mutagenesis

The first strategy for the production of a non-spliced variant used site-directed mutagenesis⁸ (Transformer TM Site-directed mutagenesis kit, Clontech, Palo Alto, Calif. USA). Two primers, Mut1 (5′-CCGCCTCGACCAAGGTGAGATATC-3′) and Mut2 (5′-CAGCATGACCCCCCAAGCCGTGCTGGCGTTC-3′) were used to introduce the desired third-base mutations (bold) at the splice donor (267) and acceptor sites (494), (bases numbered from the ATG start codon) into the HSV-TK gene contained within the pSFCMM3 vector⁹. DNA from corrected clones (p-scSFCMM3) was sequenced as described.⁷

Correction of Splice Acceptor Site by Minimal PCR

The splice acceptor site is flanked by Bgl1 sites at positions 417 and 534. A 137 base-pair fragment of DNA was amplified using primers Mut3 (5-CGTGACCGACGCCGTTCTGGCTCCT-3) and Mut4 (5-GGCCACGAACGCCAGCACGGCTTGGGG-3) (Bgl1 sites). The downstream primer includes a third-base degenerate point mutation (bold). Plasmid, pBSCK+ (Stratagene, La Jolla, Calif. USA) was digested with Bgl1, blunt ended and religated to form pBSCdBgl1. The HSV-Tk gene was removed from pSP65Tk (GTI Gaithersburg, Md., USA) using Bgl11 and Xho1 and cloned into the BamH/Xho1 sites of pBSCdBgl. This plasmid was digested with Bgl1, gel purified before ligation to the amplified Bgl1 digested HSV-Tk mutated fragment. Wild-type and corrected HSV-Tk genes were sequence-verified before, a Not1/Xho1 digestion transferred them to the retroviral vector pSF/SV/neo (a modification of the pSF1N vector¹⁰ now containing a cloning site and a neomycin phosphotransferase gene expressed from an internal SV40 promoter) to form respectively pSF/Tk/wt and pSF/Tk/mut.

Generation of Virus-Producer Cell Lines

Supernatants from p-scSCMM3 transfected GP+E86 ecotropic packaging cell¹¹ were used to infect the GP+env Am12 amphotropic cell line¹². pSF/Tk/mut and pSF/Tk/wt were transfected into Ψ crip¹³ and selected in 400 μg/ml G418 (Life-technologies, Cergy, Pontoise, France).⁷ Analysis of splicing in transduced cells.

Primary T cells and the T cell lines, HuT78 (ATCC TIB-161) and CEM (ATCC CRL 2265)] were transduced with G1Tk1SVNa, SF/Tk/wt, SF/Tk/mut, SFCMM3 or scSFCMM3 vectors. HSV-Tk PCR (as described in⁷) was performed on transduced target cells. In addition, PCR was performed using primers which selectively amplified the deleted form of the HSV-Tk gene using a 5′ primer, which spans the truncation point [TrTk1: 5′CTCGACCAGG§GCCGTGCT. (§ denoting the junction) and the previously described RTk2 3′ primer⁷. Splicing from the transduced cell lines was quantified as described.⁷

Results and Discussion

We adopted two successful approaches to eliminate splicing within the HSV-Tk gene as evidenced by the absence of the truncated HSV-Tk gene in T cells lines or primary T cells transduced with virus produced by corrected HSV-Tk containing packaging cell lines.

In FIG. 17A, a Southern blot of PCR products derived from Hut78 cells transduced with G1Tk1SVNa, SF/Tk/wt +/−GCV shows a decrease in the signal from the full-length HSV-Tk gene as well as an increased signal from the deleted gene as the concentration of GCV is increased (as also observed in vivo⁷). In contrast, the truncated gene is never detected in cells transduced with SF/Tk/mut. Identical findings were observed using CEM cells as target cells (data not shown) using SF/TK/wt, SF/Tk/mut as well as SFCMM3 and scSFCMM3 viruses.

Analysis of primary T cells transduced with SFCMM3 or scSFCMM3 vector also showed the full-length gene in cells transduced with the corrected gene whereas both full-length and truncated forms were seen in cells containing the wild-type vector (FIG. 17B). PCR using a truncation-specific primer gave similar results (FIG. 17C).

We previously reported that the frequencies of splicing events in CEM cells transduced with SCFMM3 and G1Tk1SVNa vectors were 15.5% and 9.2%, respectively.⁷ Use of corrected vectors confirmed the absence of splicing in all screened colonies (0/46 with SF/Tk/mut and 0/126 with sc-SFCMM3).

In FIG. 18, we demonstrate that the sensitivity of transduced cells to GCV has not been compromised by the mutation, as expected since the HSV-Tk amino acid sequence has been conserved. GCV sensitivity of T cell lines and primary T cells expressing the mutated HSV-Tk gene was only marginally increased compared to populations transduced by wild-type gene. This particular assay of GCV-induced inhibition of cell proliferation might not be optimal to demonstrate an increased GCV sensitivity over the 80 to 90% inhibition achieved with cells expressing the wild-type gene.¹⁴ In agreement with this hypothesis, is the finding that GCV inhibition of murine HSV-Tk-transgenic T cells¹⁵ (where both full-length and truncated messages are produced from the same wild-type HSV-Tk gene [data not shown]) is also in the 80 to 90% range.¹⁶ Ultimately, only in vivo assessment might be able to demonstrate increased GCV sensitivity.

It is possible that other mechanisms such as DNA methylation,¹⁷ post-transcriptional processing of mRNA or mutations in the three step pathway^{18,19} may provide an explanation in those cases in which intact HSV-Tk message has been detected in transduced cells.

In addition, GCV resistance by alternative mechanisms might occur in primary T cells. Despite the production of intact HSV-Tk message, transduced cells might develop GCV resistance by mechanisms such as gene silencing by DNA methylation^{17,18}. In addition, resistant T cell subsets may be avoiding the effects of GCV by a temporary withdrawal from the cell cycle¹⁹ Lastly, since GCV is metabolized by a three-step pathway,²⁰ mutations in other genes may also be involved.

Despite these reservations, GCV-resistant truncated HSV-Tk expressing cells constituted significant proportion of the circulating gene modified cells after GCV treatment in our patients.⁷ One can therefore expect that the use of a corrected HSV-Tk gene in which cryptic splicing is abolished will significantly reduce the number of GCV-resistant gene-modified donor T cells in vivo. Further clinical trials using HSV-TK expressing cells should benefit from the use of such a mutated HSV-Tk gene.

### EXAMPLE 4

### Treatment

Gene therapy is a clinical strategy in which the genome of somatic cells is modified for therapeutic purposes. Valuable information on human physiology can also be gained from gene transfer studies not directly focused in a clinical benefit for the recipient of the genetically engineered cells. Essentially, gene transfer involves the delivery to target cells of an expression vector containing one or more genes as well as the sequences required for the control of their expression. Generally, the expression vector is transferred into the target cells in vitro. Modified cells carrying the expression vector are then administered to the recipient. Recently, the in vivo administration of the expression vector to the cells within an individual is also a feasible alternative.

In the majority of the clinical trials, recombinant retroviruses are the vector systems more commonly used as vehicles for gene delivery. The genetic information is carried in the form of RNA and enters the target cell via a specific receptor. Inside the infected cell is converted into DNA by reverse-transcription. Virus-derived DNA is then randomly incorporated into the genome of the host cell (provirus) where the expression cassette start coding for the therapeutic genes.

The retrovirus vectors used in clinical gene transfer studies are derived from murine leukaemia virus (MLV). Almost all retrovirus vectors systems consist of two components. The first component is the expression vector that contains the therapeutic genes. This, in the form of RNA, constitutes the genome of the retroviral vector particle. The gag, pol, and env are deleted from the virus, rendering it repliaction-deficient. The second component of the system, the packaging cell line is required for the production of vector virus particles. These cells are engineered to produce the missing retroviral structural proteins (gag, pol, and env) from two different expression constructs (third generation retrovirus vector system). These proteins are required to package the virus able to infect (transduce) target cells.

The expression of the HSV-tk gene in genetically engineered mammalian cells makes them sensitive to the prodrug GCV. HSV-derived thymidine kinase can phosphorilate the GCV. Monophosphorylated GCV is converted by cellular kinases to GCV triphosphate which inhibits DNA replication by chain termination (FIG. 13). This strategy has been used to induce remission of transplanted tumours in various animal models (Freeman S M et al, 1996; Semin Oncol 23: 31-45). Its use is also being evaluated in the treatment of brain and kidney tumours in humans (Culver K W, Blaese R M Trends. Genet. 10: 174, 1994; Moolten F L, Wells J M: J. Natl. Cancer Inst. 82: 297, 1990).

Patients in remission or with relapsed haematologic malignances after allogeneic bone marrow transplantation are being currently infused with HSV-tk transduced donor T-lymphocytes. This approach allows the selective depletion from circulation of the alloreactive donor T-cells responsible for the graft versus host disease (GvHD) a major problem in adoptive immunotherapy (Graft-versus-hostdisease: Immunology, pathophysiology and treatment. New York, Marcel Dekker, 1990).

The vectors described herein are used in these approaches.

### EXAMPLE 5

### Identification of the Truncated HSV-tk Gene in Clinical Samples

The same molecular mechanism for ganciclovir resistance in donor human T-lymphocytes transduced with retroviral vectors carrying the Herpes simplex virus thymidine kinase gene was observed in clinical samples as in the in vitro studies described in Example 1.

Analogous studies to those in Example 1 were carried out in in vivo circulating donor T-cell transduced with another retroviral vector (G1Tk1SvNa; see FIG. 1 for a description of its structure; the vector contains the HSV-tk and Neo^{R} genes). The following summarises the findings and, where appropriate, differences in methodology compared to Example 1.

The retroviral vector G1Tk1SvNa has been used in a number of therapeutic clinical trials (Packer et al (2000) J. Neurosurg. 92, 249-254; Tiberghien et al (1996) Hematol Cell Ther. 38, 221-224. We identified the presence of the 227 bp deletion due to cryptic splicing of HSV-Tk RNA in patients who had received G1Tk1SvNa transduced donor T-cells following a T-cell depleted allo-SCT (Tiberghien et al (1996) Hematol. Cell. Ther. 38, 221-224). These observations are of particular importance for the design of vectors destined for future clinical use.

Materials and Methods

Retroviral Vectors and Producer Lines

The PA317-derived producer cell containing the G1Tk1SvNa retroviral vector was provided by Genetic Therapy, Inc. Novartis, (Gaithersburg, Md., USA) (FIG. 1). The vectors and their producer cell lines have been described previously (Tibergbien et al (1997) Hum. Gene Ther. 8, 615-624; Verzeletti et al (1998) Hum. Gene Ther. 9, 2243-2251; Lyons et al (1995) Cancer Gene Ther. 2, 273-280).

Isolation and Culture of Human T-Cell Lines and Primary T Lymphocytes

PBMNCs were collected in EDTA tubes. Cells were cultured with 1000 U/ml penicillin for day 0 to 3, the 500 U/ml rhIL-2.

Transduction of donor T-cells with the G1Tk1SvNa retroviral vector was as previously described (Robinet et al (1998) J. Hematother. 7, 205-215). PBMNC were cultured for 3 days with OKT3 (10 ng, Ortho) and rhIL-2 (500 U/mL, Chiron). Infection with retroviral supernatant for 24 hours was followed by G418 (800 μg/mL, Sigma) selection of transduced cells for 7 days. Dead cells were removed and the viable transduced product was cryopreserved for future use. The transduction of CEM cells with the G1Tk1SvNa vector involved identical infection conditions to those described for the SFCMM3 vector, followed by selection in G418 (800 μg/ml) for 7-14 days.

Cells transduced with G1Tk1SvNa were selected by exposure to G418 (800 μg/ml) for 7-14 days. The transduction efficiency was evaluated by a competitive PCR assay for the Neo^{R} gene.

Polymerase Chain Reaction and Sequence Analysis

The presence of the HSV-Tk gene in G1Tk1SvNa transduced cells was examined by PCR using primers (FIG. 1B): pTK (5′ TAGACGGTCCTCACGGGATGGGGA 3′) and RTK2 (5′ GCCAGCATAGCCAGGTCAAG 3′). PCR assays were performed in a 50 μl reaction mixture containing 500 ng of DNA, 1×Taq polymerase buffer with 15 mM MgCl₂ (Eurogentec, Seraing, Belgium), 200 μM each dNTP, 0.25 μM each primer, and 0.5 U of Taq DNA polymerase (Eurogentec). Thermocycling conditions were 35 cycles of 94° C. for 45 seconds, 60° C. for minute, 72° C. for 1 minute, followed by a final extension of 5 minutes at 72° C. PCR products (18 μl) were electrophoresed on a 2% agarose gels EtBr stained. Genomic DNA transfer to nylon filter was performed using standard conditions. The blots were hybridized with an α-³²P-dCTP end tailing-labelled oligoprobe: Tk2 probe (5′ATCGTCTACGTACCCGAGCCGATGA 3′), and washed at 60° C. in 0.1×SCC−0.1% SDS before autoradiography. The sensitivity of this assay, determined by amplification of diluted DNA extracted from the packaging cell line allowed the detection of one transduced cell in 10⁵ unmodified cells, assuming that there is one TK gene copy per genome (Brodie et al (1999) Nat. Med. 5, 34-41).

PCR products were cloned in duplicate into the pGEMT Easy vector from Promega (Charbonnieres, France). Automated fluorescent DNA sequence analysis was carried out by PE Biosystems (COURTABOEUF, France).

Frequency of Truncation Event

Three hundred CEM cells which had been transduced by either G1Tk1SVNa or SFCMM3 vectors and selected by G418 or immunomagnetic procedures were mixed with 150 μl of RF10 to 1 mL of methyl cellulose (MethoCult H4320; Stem Cell Technologies, Vancouver, Canada). Cells were then plated in triplicate in 35 mm Petri dishes and grown for 15 days at 37° C. in 5% CO₂ and scored on day 15. Individual colonies were picked and expanded in 24 well plates for 3 days. Cells were then harvested for analysis by PCR for the presence of full-length or truncated Tk gene as described above.

Clinical Study

Escalating amounts of CD3⁺ gene-modified cells were infused with T cell depleted bone marrow. Twelve patients with hematological malignancies received 2×10⁵ (n=5), 6×10⁵ (n=5) or 20×10⁵ (n=2) transduced donor CD3⁺ cells/kg with bone marrow from an HLA-identical sibling. Acute toxicity was not observed. Quantititive PCR for Neo^{R} revealed an early increase of the circulating transduced T-cells followed by a progressive decrease over time with, however, persisting detectable long-term (more than 800 days) circulation of gene-modified cells. Three patients developed acute GvHD grade≧II while 1 patient developed chronic GvHD (skin and salivary glands). Treatment with GCV alone was associated with a complete remission (CR) in 2 patients with acute GvHD while addition of steroids was necessary to achieve a CR in the last case. Long-lasting CR was associated with GCV treatment in the patient with chronic GvHD. Ganciclovir treatment resulted in a significant rapid decrease in circulating transduced T-cells. In patients receiving GCV, blood samples for detection of gene-modified cells expressing the wild-type or truncated form of the HSV-Tk gene were harvested prior to GCV treatment and a regular two weeks intervals.

Results

Transduction Efficiency of Primary Cells

The transduction efficiency of primary T-cells with G1Tk1SvNa prior to G418 selection, as measured by quantitative PCR for Neo^{R}, was 8.3±1.4% (n=12). The GCV sensitivity of these cells following G418 selection was 87±1.2% (n=12). The relative cell growth of control and transduced cells from one representative transduction/selection experiment is shown in FIG. 16.

PCR and Sequence Analysis of Transduced Primary T-lymphocytes

Genomic DNA extracted from human primary T-lymphocytes transduced with the G1Tk1SvNa vector was also analysed by PCR for the HSV-Tk gene. In this study two bands were identified in both selected (TK800) and unselected transduced cells (TK0) (FIG. 14A). The 560 bp fragment corresponds to the full-length HSV-Tk gene and gave the strongest signal, compared to the smaller band of 333 bp corresponding to the deleted form of the HSV-Tk gene. This may be due to competition in the PCR assay between the full-length and truncated Tk forms, when the target present in the larger amount is preferentially amplified. However, when transduced cells were first selected in G418 and then cultured in the presence of GCV (TK800+GCV), the 333 bp band became more intense. This indicates that GCV treatment resulted in the enrichment of a population of cells expressing the truncated HSV-Tk gene (FIG. 14B). Interestingly, sequencing of the 333 bp PCR product showed the exact same deletion of 227 bp within the HSV-Tk sequence (donor and acceptor sites at position 1871 and 2098 respectively in the G1Tk1SvNa vector) as in the CEM and primary T-cells transduced with the SCFMM3 vector (see Example 1).

Frequency of Truncation Event

With the G1Tk1SvNa vector, PCR analysis of 153 CEM transduced colonies showed that 14 contained the truncated form of the HSV-Tk gene while 139 colonies had the full-length HSV-TK gene giving a truncation frequency of 9.15%. 45 colonies transduced with the SFCMM3 vector were analysed by PCR and 7 were found to contain the truncated gene (frequency 15.5%).

Identification of the Truncated HSV-Tk Gene in Clinical Samples

In vivo circulating transduced T-cells containing the truncated form of the HSV-Tk gene were observed in all 12 patients as early as 30 minutes after infusion up to 800 days after transplantation. Although quantification of both forms of the HSV-Tk, gene were not performed, PCR followed by Southern blot analysis suggested that the proportion of in vivo transduced T-cells expressing the truncated HSV-Tk gene, was present in a proportion similar to or less than that observed in vitro (<10%)(FIG. 15, lane A). As illustrated in a representative sample from one patient (FIG. 15), GCV administered as treatment for GvHD resulted in a decrease of the signal generated by the full-length HSV-Tk gene, whereas the signal associated with the truncated form of the HSV-Tk gene progressively increased. Importantly, GCV treatment always significantly reduced the percentage (85-98%) and absolute number (76-99.5%) of circulating transduced T-cells as determined by quantitative PCR of the Neo^{R} gene¹⁷. Thus, on increasing proportion of transduced T-cells containing the truncated HSV-Tk gene was present in a reduced number of circulating transduced cells.

Conclusion

We confirmed that the 227 bp deletion in the HSV-Tk gene due to the presence of cryptic splice donor and acceptor sites was present in 12 patients that received transduced donor T-cells together with a T-cell depleted allo-SCT. In vivo circulating transduced T-cells containing the truncated HSV-Tk gene were identified in all patients immediately after infusion and up to 800 days after transplantation. In patients who received GCV as treatment for GvHD, a progressive increase in the proportion of transduced donor T-cells carrying the deleted HSV-Tk gene was observed. These results suggest that the limitations within the HSV-Tk/GCV system can be improved by developing optimised retroviral vectors to ensure maximal killing of HSV-Tk transduced cells.

### REFERENCES FOR EXAMPLE 1

1 Culver K W, Blaese R M (1994) “Gene therapy for cancer” Trends. Genet. 10, 174.

2. Moolten F L, Wells J M (1990) “Curability of tumors bearing herpes thymidine kinase genes transferred by retroviral vectors” J. Natl. Cancer Inst. 82, 297.

3. Moolten F L (1994) “Drug sensitivity (“suicide”) genes for selective cancer chemotherapy” Cancer Gene Ther. 1, 279.

4. Moolten F L, Wells J M (1986) “Tumor chemosensitivity conferred by inserted herpes thymidine kinase genes: paradigm for a prospective cancer control strategy” Cancer Res. 46, 5276.

5. Horowitz M M, Gale R P, Sondel P M, Goldman J M, Kersey J, Kolb H J, Rimm A A, Ringden O, Rozman C, Speck B, et al (1990) “Graft-versus-leukemia reactions after bone marrow transplantation” Blood 75, 555.

6. “Anonymous Graft-versus-host-disease: Immunology, pathophysiology and treatment” in Burakoff, S J, Degg, H J, Ferrara, J, Atkinson, K (eds): Hematology, New York, Marcel Dekker (1990).

7. Kolb H J, Schattenberg A, Goldman J M, Hertenstein B, Jacobsen N, Arcese W, Ljungman P, Ferrant A, Verdonck L, Niederwieser D, et al (1995) “Graft-versus-leukemia effect of donor lymphocyte transfusions in marrow grafted patients. European Group for Blood and Marrow Transplantation Working Party Chronic Leukemia” [see comments] Blood 86, 2041.

8. Bordignon C, Bonini C, Verzeletti S, Nobili N, Maggioni D, Traversari C, Giavazzi R, Servida P, Zappone E, Benazzi E, et al (1995) “Transfer of the HSV-tk gene into donor peripheral blood lymphocytes for in vivo modulation of donor anti-tumor immunity after allogeneic bone marrow transplantation” Hum. Gene Ther. 6, 813.

9. Tiberghien P, Cahn J Y, Brion A, Deconinck E, Racadot E, Herve P, Milpied N, Lioure B, Gluckman E, Bordigoni P, Jacob W, Chiang Y, Marcus S, Reynolds C, Longo D (1997) “Use of donor T-lymphocytes expressing herpes-simplex thymidine kinase in allogeneic bone marrow transplantation: a phase I-II study” Hum. Gene Ther. 8, 615.

10. Bonini C, Ferrari G. Verzeletti S, Servida P, Zappone E, Ruggieri L, Ponzoni M, Rossini S, Mavilio F, Traversari C, Bordignon C (1997) “HSV-TK gene transfer into donor lymphocytes for control of allogeneic graft-versus-leukemia” [see comments] Science 276, 1719.

11. DiMaio J M, Clary B M, Via D F, Coveney E, Pappas T N, Lyerly H K (1994) “Directed enzyme pro-drug gene therapy for pancreatic cancer in vivo” Surgery 116, 205.

12. Mavilio F, Ferrari G, Rossini S, Nobili N, Bonini C, Casorati G, Traversari C, Bordignon C (1994) “Peripheral blood lymphocytes as target cells of retroviral vector-mediated gene transfer” Blood 83, 1988.

13. Febse B, Schade U M, Li Z, Ubde A, Koch S, Goller B, Ruger R, Fehse N, Stockschlader M, Zander A R (1998) “Highly-efficient gene transfer with retroviral vectors into human T lymphocytes on fibronectin” Br. J. Haematol. 102, 566.

14. Rudoll T, Phillips K, Lee S W, Hull S, Gaspar O, Sucgang N, Gilboa E, Smith C (1996) “High-efficiency retroviral vector mediated gene transfer into human peripheral blood CD4+ T lymphocytes” Gene Ther. 3, 695.

15. Freeman S M, Abboud C N, Whartenby K A, Packman C H, Koeplin D S, Moolten F L, Abraham G N (1993) “The “bystander effect”: tumor regression when a fraction of the tumor mass is genetically modified” Cancer Res. 53, 5274.

16. Ishii M H, Agbaria R, Mullen C A, Hirano H, Koeplin D A, Ram Z, Oldfield E H, Johns D G, Blaese R M (1997) “Mechanism of ‘bystander effect’ killing in the herpes simplex thymidine kinase gene therapy model of cancer treatment” Gene Ther. 4, 244.

17. Tiberghien P, Reynolds C W, Keller J, Spence S, Deschaseaux M, Certoux J M, Contassot E, Murphy W J, Lyons R, Chiang Y, et al (1994) “Ganciclovir treatment of herpes simplex thymidine kinase-transduced primary T lymphocytes: an approach for specific in vivo donor T-cell depletion after bone marrow transplantation?” Blood 84, 1333.

18. Moolten F L, Wells J M, Heyman R A, Evans R M (1990) “Lymphoma regression induced by ganciclovir in mice bearing a herpes thymidine kinase transgene” Hum. Gene Ther. 1, 125.

19. Barba D, Hardin J, Ray J, Gage F H (1993) “Thymidine kinasemediated killing of rat brain tumors” J. Neurosurg. 79, 729.

20. Di I M, Casciari C, Ciurnelli R, Fulvi A, Bagnis C, Sadelain M, Lucheroni F, Mannoni P, Stella C C, Martelli M F, Tabilio A (1997) “Retroviral transfer of herpes simplex virus-thymidine kinase and beta-galactosidase genes into U937 cells with bicistronic vector” Leuk. Res. 21, 951.

21. Yang L, Hwang R, Chiang Y, Gordon E M, Anderson W F, Parekh D (1998) “Mechanisms for ganciclovir resistance in gastrointestinal tumor cells transduced with a retroviral vector containing the herpes simplex virus thymidine kinase gene” Clin. Cancer Res. 4, 731.

22. Sorrentino B P, McDonagh K T, Woods D, Orlic D (1995) “Expression of retroviral vectors containing the human multidrug resistance 1 cDNA in hematopoictic cells of transplanted mice” Blood 86, 491.

### REFERENCES FOR EXAMPLE 3

1. Tiberghien P, Reynolds C W, Keller J, et al: Ganciclovir treatment of herpes simplex thymidine kinase-transduced primary T-lymphocytes: an approach for specific in vivo donor T-cell depletion after bone marrrow transplantation Blood (1994) 84, 1333-1341.

2. Bonini C, Ferrari G, Verzeletti S, et al.: HSV-Tk gene transfer into donor lymphocytes for control of allogeneic graft-versus-leukaemia. Science (1997) 276, 1719-1724.

3. Tiberghien P, Ferrand C, Lioure B, et al: Administration of herpes simplex-thymidine kinase-expressing donor T cells with a T-celldepleted allogeneic marrow graft. Blood (2001) 97, 63-72.

4. Tiberghien P, “Suicide” gene for the control of graft-versus-host disease. Current opinions in Haematology (1998) 5, 478-482.

5. Champlin R, Bensinger W, Henslee-Downey J, et al: A phase I/II study of Thymidine kinase (Tk)-transduced donor lymphocyte infusions (DLI) in patients with haematological malignancies. Blood (1999) 94, Supplement 1, Abstract 1448.

6. Link C J, Drobyski W R, Traynor A E, et al: Adoptive immunotherapy for leukaemia:donor lymphocytes transduced with the herpes simplex thymidine kinase (HSV-Tk). Blood (1998) 94: Supplement1, Abstract 1631.

7. Garin M I, Garrett E, Tiberghien P, et al: Molecular mechanism for ganciclovir resistance in human T-lymphocytes transduced with retroviral vectors carrying the herpes simplex virus thymidine kinase gene. Blood (2001) 97, 122-129.

8. Deng W P, Nickoloff J A. Site-directed mutagenesis of virtually any plasmid by eliminating a unique site. Anal. Biochem. (1992) 200, 81-88.

9. Verzeletti S, Bonini C, Marktel S, et al: Herpes simplex virus thymidine kinase gene transfer for controlled graft-versus-host-disease and graft-versus-leukemia: clinical follow-up and improved new vectors. Hum. Gene Ther. (1998) 9, 2243-2251.

10. Baum C, Eckert H G, Stockschlader M S, et al: Improved retroviral vectors for haemopoietic stem cell protection and in vivo selection. J. Hematother. (1996) 5, 323-329.

11. Markowitz D, Goff S, Bank, A. A safe packaging cell line for gene transfer: Separating viral genes on two different plasmids. J. Virol. (1988) 62, 111120-11124.

12. Markowitz D, Goff S, Bank A. Construction and use of a safe an efficient amphotropic packaging cell line. Virology (1988) 167, 400-406.

13. Miller A D. Retrovims packaging cells. Hum. Gene Ther. (1990) 1, 5-14.

14. Contassot E, Ferrand C, Certoux J M, et al: Retrovirus-mediated transfer of the herpes simplex type I thymidine kiase gene in alloreactive T lymphocytes. Hum. Gene Ther (1998) 9, 73-80.

15. Cohen J L, Boyer O, Salomon B, et al: Fertile homozygous transgenic mice expressing a functional truncated herpes simplex thymidine kinase delta TK gene. Transgenic Res. (1998) 5, 321-330.

16. Contassot E, Ferrand C, Angonin R, et al: Gacnciclovir-sensitive acute graft-versus-host disease in mice receiving herpes simplex virus-thymidine kinase-expressing donor T cells in a bone marrow transplantation setting. Transplantation (2000) 69, 503-4.

17. Degreve B, De Clecq E, Balazarini J. et al: Selection of HSV-1 TK gene-transfected murine mammary carcinoma cells resistant to (E)-5-(2-bromovinyl)-2′-deoxyuridine (BVDU) and ganciclovir. Gene Ther. (2000) 18, 1543-1552.

18. Di lanni M, Terenzi A, Di Florio S, et al: In vivo demethylation of a MoMuLV retroviral vector expressing the herpes simplex thymidine kinase suicide gene by 5′ azacytidine. Stem cells (2000) 18, 415-421.

19. Golumbek P T, Hamzab F M, Levitsky H, Lietman P S, Pardoll D M: Herpes simplex-1 virus thymidine kinase gene is unable to completely eliminate live, non-immunogenic tumor cell vaccines. J. Immunother. (1992) 12, 224-230.

20. Faulds D, Heel R C. Ganciclovir. A review of its antiviral activity, pharmacokinetic properties and therapeutic efficacy in cytomegalovirus infections. Drugs (1990) 39, 597-638.

1. A polynucleotide encoding a thymidine kinase wherein the thymidine kinase coding region does not contain a functional splice acceptor and/or splice donor site.

2. A polynucleotide according to claim 1 wherein the thymidine kinase is a Herpesviridae thymidine kinase.

3. A polynucleotide according to claim 2 wherein the thymidine kinase is a herpes simplex virus thymidine kinase.

4. A polynucleotide according to claim 3 wherein the thymidine kinase is herpes simplex virus type 1 thymidine kinase.

5. A polynucleotide according to any one of the preceding claims wherein the thymidine kinase coding region encodes wild type thymidine kinase.

6. A polynucleotide according to any one of claims 1 to 4 wherein the thymidine kinase coding region encodes thymidine kinase which, when compared to wild type, contains mutations that enhance the enzymatic activity.

7. A polynucleotide according to claim 4 wherein compared to the wild type sequence any one of the nucleotides at positions 842 and 1070 in FIG. 1I is replaced by another nucleotide such that there is no splice site present.

8. A polynucleotide according to claim 7 wherein compared to the wild type sequence nucleotide the splice donor site at position 842 is changed from GAC CAG GGT to GAC CAA GGT.

9. A polynucleotide according to claim 7 wherein compared to the wild type sequence nucleotide the splice acceptor at position 1070 is changed from CCC CAG GCC to CCC CAA GCC.

10. A polynucleotide according to claim 6 wherein compared to the wild type sequence nucleotide the splice donor at position 842 is changed from GAC CAG GGT to GAC CAA GGT and the splice acceptor at position 1070 is changed from CCC CAG GCC to CCC CAA GCC.

11. A polynucleotide according to any one of the preceding claims further comprising a promoter in operable linkage to allow for expression of the coding region.

12. An expression vector comprising a polynucleotide as defined in any one of the preceding claims.

13. An expression vector according to claim 12 which is a viral vector.

14. An expression vector according to claim 13 which is a retroviral vector.

15. An expression vector according to claims 12 to 14 adapted for delivery to a patient.

16. A host cell comprising a polynucleotide according to claims 1 to 11 or an expression vector according to claims 12 to 15.

17. A pharmaceutical composition comprising a polynucleotide according to any one of claims 1 to 11, or an expression vector according to any one of claims 12 to 15, and a pharmaceutically acceptable carrier.

18. A polynucleotide according to any one of claims 1 to 11, or an expression vector according to any one of claims 12 to 15, for use in medicine.

19. A method of destroying cells the method comprising introducing into the cells a polynucleotide according to any one of claims 1 to 11 or an expression vector according to any one of claims 12 to 15, allowing the cells to express thymidine kinase, and contacting the cells with a substantially non-toxic agent which is converted by thymidine kinase to a toxic agent.

20. A method of treating a patient with cells in need of destruction the method comprising introducing into the patient a polynucleotide according to any one of claims 1 to 11 or an expression vector according to any one of claims 12 to 15, allowing the polynucleotide or expression vector to be taken up by the cells, allowing the cells to express thymidine kinase, and administering to the patient a substantially non-toxic agent which is converted by thymidine kinase to a toxic agent.

21. A method of treating a patient with cells in need of destruction, the method comprising (1) removing the cells from the patient or donor of cells, (2) introducing into the cells ex vivo a polynucleotide according to claims 1 to 11 or an expression vector according to claims 12 to 15, (3) introducing the modified cells into the patient which may or may not be expressing thymidine kinase when so introduced, (4) optionally, allowing the cells to express thymidine kinase if not so expressing and (5) administering to the patient a substantially non-toxic agent which is converted by thymidine kinase into a toxic agent.

22. A method according to claims 19 to 21 wherein the substantially non-toxic agent is any one of ganciclovir, acyclovir, trifluorothymidine, 1-[2-deoxy,2-fluoro,β-D-arabino furanosyl]-5-iodouracil, ara-A, ara 1,1-β-D arabino furanosyl thymine, 5-ethyl-2′-deoxyuridine, 5-iodo-5′-amino-2,5′-dideoxyuridine, idoxuridine, AZT, AIV, dideoxycytidine, Ara C and bromovinyl deoxyuridine (BVDU).

23. Use of a polynucleotide according to any one of claims 1 to 11 or an expression vector according to any one of claims 12 to 15 in the manufacture of a medicament for destroying cells in a patient wherein the patient has been, is being or will be administered a substantially non-toxic agent which is converted by thymidine kinase to a toxic agent.

24. Use of a substantially non-toxic agent which is converted by thymidine kinase to a toxic agent in the manufacture of a medicament for destroying cells in a patient wherein the patient has been, is being or will be administered with a polynucleotide according to any one of claims 1 to 11 or an expression vector according to any one of claims 12 to 15.

25. A therapeutic system comprising a polynucleotide according to any one of claims 1 to 11 or an expression vector according to any one of claims 12 to 15 and a substantially non-toxic agent which is converted by thymidine kinase to a toxic agent.

26. The use of claims 23 or 24 or the system of claim 25 wherein the substantially non-toxic agent is any one of ganciclovir, acyclovir, trifluorothymidine, 1-[2-deoxy,2-fluoro,β-D-arabino furanosyl]-5-iodouracil, ara-A, ara 1,1-β-D arabino furanosyl thymine, 5-ethyl-2′-deoxyuridine, 5-iodo-5′-amino-2, 5′-dideoxyuridine, idoxuridine, AZT, AIV, dideoxycytidine, Ara C and bromovinyl deoxyuridine (BVDU).

27. A method of making a polynucleotide according to claim 1 the method comprising (1) determining whether a natural coding region for the thymidine kinase contains a functional splice acceptor and/or splice donor site and (2) if it does, mutating at least one of the splice acceptor and/or splice donor sites to make them non-functional.

28. A method according to claim 27 wherein step (1) comprises analysing mRNA transcribed from at least part of the natural coding region.

29. A method according to claim 27 or 28 wherein in step (2) a site-direct mutagenesis is used to introduce the mutation.

30. Any novel gene therapy vector or system as herein disclosed.


SEQUENCE LISTING (20 sequences)

SEQ ID NO: 1 - DNA, 23 bp, Artificial Sequence
  Description of Artificial Sequence Primer
ggtctcctctgagtgattgacta

SEQ ID NO: 2 - DNA, 23 bp, Artificial Sequence
  Description of Artificial Sequence Primer
aacgaattccggcgcctagagaa

SEQ ID NO: 3 - DNA, 23 bp, Artificial Sequence
  Description of Artificial Sequence Primer
ttctctaggcgccggaattcgtt

SEQ ID NO: 4 - DNA, 23 bp, Artificial Sequence
  Description of Artificial Sequence Primer
atccaggataaagacgtgcatgg

SEQ ID NO: 5 - DNA, 23 bp, Artificial Sequence
  Description of Artificial Sequence Primer
ccatgcacgtctttatcctggat

SEQ ID NO: 6 - DNA, 23 bp, Artificial Sequence
  Description of Artificial Sequence Primer
ttgcagcactcaccgctgtgtgt

SEQ ID NO: 7 - DNA, 23 bp, Artificial Sequence
  Description of Artificial Sequence Primer
acacacagcggtgagtgctgcaa

SEQ ID NO: 8 - DNA, 23 bp, Artificial Sequence
  Description of Artificial Sequence Primer
atagaaggcgatgcgctgcgaat

SEQ ID NO: 9 - DNA, 24 bp, Artificial Sequence
  Description of Artificial Sequence Primer
ccgcctcgaccaaggtgagatatc

SEQ ID NO: 10 - DNA, 31 bp, Artificial Sequence
  Description of Artificial Sequence Primer
cagcatgaccccccaagccgtgctggcgttc

SEQ ID NO: 11 - DNA, 24 bp, Artificial Sequence
  Description of Artificial Sequence Primer
agtgcaccatgggcggtgtgaaat

SEQ ID NO: 12 - DNA, 27 bp, Artificial Sequence
  Description of Artificial Sequence Primer
ggccacgaacgccagcacggcttgggg

SEQ ID NO: 13 - DNA, 18 bp, Artificial Sequence
  Description of Artificial Sequence Primer
ctcgaccagggccgtgct

SEQ ID NO: 14 - DNA, 24 bp, Artificial Sequence
  Description of Artificial Sequence Primer
tagacggtcctcacgggatgggga

SEQ ID NO: 15 - DNA, 20 bp, Artificial Sequence
  Description of Artificial Sequence Primer
gccagcatagccaggtcaag

SEQ ID NO: 16 - DNA, 25 bp, Artificial Sequence
  Description of Artificial Sequence Primer
atcgtctacgtacccgagccgatga

SEQ ID NO: 17 - DNA, 1799 bp, herpes simplex virus 7
gggtcctaggctccatggggaccgtatacgtggacaggctctggagcatcgcacgactgcgtgatattaccggagaccttctgcgggacgagccgggtcacgcggctgacggagcgtccgttgggcgacaaacaccaggacggggcacaggtacactatcttgtcacccggagcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatcttggtggcgtgaaactcccgcacctctttggcaagcgccttgtagaagcgcgtatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatgtcgggggggaggctgggagttcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggg

SEQ ID NO: 18 - DNA, 1131 bp, herpes simplex virus 7
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatgtcgggggggaggctgggagttcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactga

SEQ ID NO: 19 - DNA, 904 bp, herpes simplex virus 7
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactga

SEQ ID NO: 20 - DNA, 25 bp, Artificial Sequence
  Description of Artificial Sequence Primer
cgtgaccgacgccgttctggctcct